(19) United States
(12) Patent Application Publication
(10) Pub. No.: US 2025/0122150 A1
(43) Pub. Date: Apr. 17, 2025

(54) CARBAZATE-FUNCTIONAL COMPOUND

(71) Applicant: PPG Industries Ohio, Inc., Cleveland, OH (US)

(72) Inventors: Michael Allen Mayo, Pittsburgh, PA (US); Jing Xiao, Sewickley, PA (US); Allison Brooks Domhoff, Pittsburgh, PA (US); Jonathan Garrett Weis, Allison Park, PA (US); Hongying Zhou, Allison Park, PA (US); Anand Kumar Atmuri, Wexford, PA (US)

(73) Assignee: PPG Industries Ohio, Inc., Cleveland, OH (US)

(21) Appl. No.: 18/718,098

(22) PCT Filed: Dec. 6, 2022
(86) PCT No.: PCT/US2022/080958

§ 371 Date: Jun. 10, 2024

Related U.S. Application Data
(60) Provisional application No. 63/288,074, filed on Dec. 10, 2021.

(51) Int. Cl. C07C 281/04; C09D 4/00; C09D 187/00

(52) U.S. Cl. CPC C07C 281/04; C09D 4/00; C09D 187/00

(57) ABSTRACT

A carbazate-functional compound includes a plurality of groups having the following structure: [formula (I)] where X forms at least a portion of: a urethane linkage, an ester linkage, or an ether linkage. At least one R₁ from the plurality of groups is free of a hydroxyl-functional group.

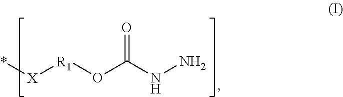

### CROSS-REFERENCE TO RELATED APPLICATIONS

This application claims the benefit of priority of U.S. Provisional Application 63/288,074 filed Dec. 10, 2021, under 35 U.S.C. 119, titled “Carbazate-Functional Compound”, which is incorporated herein by reference.

### FIELD

The present disclosure relates to a carbazate-functional compound and coating composition comprising the same.

### BACKGROUND

Coatings are applied to a wide variety of substrates to provide color and other visual effects, corrosion resistance, abrasion resistance, chemical resistance, and the like.

Many automotive original equipment manufacturer (OEM) coating compositions, such as automotive basecoats, are curable at temperatures greater than 120° C., and it is difficult to achieve good curing at lower temperatures of 100° C. or less. Moreover, certain materials used in automotive components and coated with coating compositions cannot withstand curing at the higher temperatures without deforming, distorting, or otherwise degrading.

### SUMMARY

The present disclosure is directed to a carbazate-functional compound including a plurality of groups having the following structure:

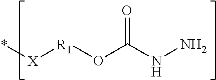

where X forms at least a portion of a urethane linkage, an ester linkage, or an ether linkage, where at least one R₁ from the plurality of groups is free of a hydroxyl-functional group.

### DETAILED DESCRIPTION

For the purposes of the following detailed description, it is to be understood that the disclosure may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses the singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise. For example, “a” polymer, “an” acid, and the like refer to one or more of any of these items.

Unless otherwise indicated, ambient conditions of temperature and pressure are ambient temperature (20-25° C.) and standard pressure of 101.3 kPa (1 atm).

As used herein, a “film-forming resin” refers to a resin forming a self-supporting continuous film on at least a horizontal surface of a substrate upon removal of any diluents or carriers present in the composition or upon curing. Also, as used herein, the term “polymer” or polymeric” is meant to refer to macromolecular compounds, i.e., compounds having a relatively high molecular mass (e.g., 500 Da or more), the structure of which comprises multiple repetition units derived, actually or conceptually, from chemical species of relatively lower molecular mass, and includes as nonlimiting examples prepolymers, oligomers, and both homopolymers and copolymers. The term “resin” is used interchangeably with “polymer”. The term “monomer” or “monomeric” is meant to refer to a compound which can contribute constitutional units to the structure of a polymer.

As used herein, the transitional term “comprising” (and other comparable terms, as nonlimiting examples, “containing” and “including”) is “open-ended” and open to the inclusion of unspecified matter. Although described in terms of “comprising”, the terms “consisting essentially of” and “consisting of” are also within the scope of the disclosure.

As used herein, the terms “on”, “applied on/over”, “formed on/over”, “deposited on/over”, “overlay”, “provided on/over”, and the like mean applied, formed, overlaid, deposited, or provided on but not necessarily in contact with the surface. For example, a coating layer “applied over” a substrate does not preclude the presence of one or more other coating layers of the same or different composition located between the formed coating layer and the substrate.

One species of crosslinker that may be used in OEM coating compositions are carbazate-functional crosslinkers which react with ketone groups of the binder of the coating compositions. The carbazate-functional crosslinkers can be made by making a carbonate-functional resin and post-reacting that resin with hydrazine. Producing the carbazate-functional crosslinkers in this way can make it difficult to control the molecular weight of the crosslinker due to the high reactivity of the dual functional hydrazine. Further, the use of hydrazine in the final step of crosslinker synthesis can disadvantageously result in higher levels of hydrazine in the resulting crosslinker composition or necessitate difficult and expensive purification steps.

The present disclosure is directed to a carbazate-functional compound that includes a plurality of groups (represented within the brackets [ ]) including the following structure (I):

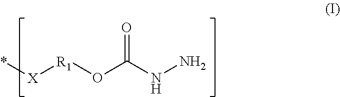

where X forms at least a portion of a urethane linkage, an ester linkage, or an ether linkage, where at least one R₁ from the plurality of groups is free of a hydroxyl-functional group.

The present disclosure also provides, as nonlimiting examples:
a hydroxyl-functional blocked carbazate compound including a blocked carbazate-functional group; and a hydroxyl-functional group;
a blocked carbazate compound including a blocked carbazate-functional group, and a linkage, X, where X forms at least a portion of a urethane linkage;
a blocked carbazate compound including a blocked carbazate-functional group, and a linkage, X, where X forms at least a portion of an ester linkage;
a blocked carbazate compound including a blocked carbazate-functional group, and a linkage, X, where X forms at least a portion of an ether linkage; and
a compound formed by reacting a carbonate with hydrazine to form an adduct; reacting the adduct with a ketone and/or aldehyde to form a hydroxyl-functional blocked carbazate; and
reacting the hydroxyl-functional blocked carbazate (1) with an isocyanate-functional compound, (2) with an anhydride-functional compound, (3) with an epoxy-functional compound, (4) with an aminoplast linkage, to form a blocked carbazate-functional compound and/or (5) in a Michael addition reaction to form a blocked carbazate-functional compound.

The carbazate-functional compound that includes Structure (I) may include a monomer, oligomer, polymer, and/or other suitable moiety.

The * of the carbazate-functional compound that includes Structure (I) indicates a bonding site at which the group of Structure (I) is bonded, and Structure (I) may be bonded to any suitable resin backbone which may include a macromolecule, oligomer, or polymer to form the carbazate-functional compound. Non-limiting examples of the compound or backbone to which the group Structure (I) may be bonded include an acrylic, a polyurethane, a polyester, a polyether, a vinyl resin, and mixtures or combinations thereof.

The plurality of groups of Structure (I) of the same the carbazate-functional compound may be the same (e.g., that include identical X and R₁) or different (e.g., that include a different X and/or R₁). At least one R₁ from the plurality of groups of Structure (I) of the carbazate-functional compound is free of a hydroxyl-functional group. The carbazate-functional compound may include at least two groups or at least three groups. The carbazate-functional compound may include two groups or three groups. The carbazate-functional compound or backbone molecule can include one or more bonding sites *, and can be, as a nonlimiting example, 2-50, such as 2-40 or 2-30 bonding sites * in the carbazate-functional compound or backbone molecule. Alternatively, as a nonlimiting example, the carbazate-functional compound or backbone molecule may have a carbazate equivalent weight of from 200 to 10,000, such as 200 to 7,500 or 200 to 5,000, calculated based on the number of equivalents of hydroxy functional carbazate.

The carbazate-functional compound including groups of Structure (I) may be both carbazate-functional and hydroxyl-functional.

As used herein, a “carbazate-functional” compound refers to a compound having a carbazate functional group or linkage:

| Carbazate Functional Group | Carbazate Linkage |
|---|---|
| 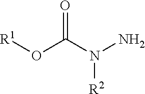 | 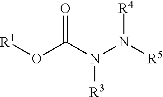 |

where any “R” group of the carbazate functional group or linkage (e.g., R¹-R⁵) refers to any suitable atom, molecule, or polymer chain unless specifically indicated otherwise, and where the R groups may be the same or different from one another. The suitable atom may include a hydrogen atom or any other suitable atom or group. As used herein, “any suitable atom or group” can include, without limitation, R¹ as described herein, R² and R³ as described herein including, but not limited to H, alkyl, alkylene, alkenyl or aryl); R⁴ and R⁵ H and deuterium. A “blocked” carbazate-functional group refers to a compound that includes a carbazate-functional group that has been blocked by a blocking agent, rendering the carbazate-functional group non-reactive under certain conditions, which blocking agent may be removed to expose the carbazate-functional group under other conditions such that the carbazate-functional group is reactive under conditions described herein.

As used herein, a “urethane linkage” refers to:

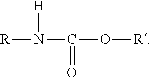

As used herein, an “ester linkage” refers to:

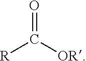

As used herein, an “ether linkage” refers to:

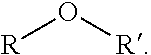

In the above structures for the urethane linkage, ester linkage and ether linkage, each R and R′ independently refer to the same or different alkyl, aryl, alkenyl, polymeric or prepolymer substituent.

As nonlimiting examples:
The X from Structure (I) may form at least a portion of a urethane linkage;
The X from Structure (I) may form at least a portion of an ester linkage; and/or
The X from Structure (I) may form at least a portion of an ether linkage.

The carbazate-functional compound includes a plurality of the groups having Structure (I) each of which may be the same or different from one another. For the carbazate-functional compound, at least one of the groups having Structure (I) can include an R₁ substituent that is free of a hydroxyl-functional group.

The X from the Structure (I) groups of the carbazate-functional compound may be the same or different from one another. As a nonlimiting example, each X from the groups may form at least a portion of a urethane linkage, an ester linkage, or an ether linkage. Alternatively and nonlimiting, one X group may form at least a portion of a urethane linkage while another X of the groups forms at least a portion of an ester linkage or an ether linkage. Any combination of Xs of the groups are within the scope of the disclosure, such that the carbazate-functional compound may be urethane functional, ester functional, ether functional, or some combination thereof.

As a nonlimiting example, from Structure (I), R₁ may be a residue of a carbonate. R₁ being a “residue” of carbonate refers to R₁ that includes material from the carbonate which remains after the carbonate undergoes a chemical reaction, such as a reaction of carbonate with hydrazine. The carbonate may be a cyclic carbonate. Non-limiting examples of cyclic carbonates include ethylene carbonate, propylene carbonate, trimethylene carbonate, glycerol carbonate, or some combination thereof. The cyclic carbonate may include a series of atoms connected to form a ring. A cyclic carbonate includes a hydroxyl group, such as glycerol carbonate. The cyclic carbonate may have a five-membered or a six-membered ring. The carbonate may be non-cyclic, such as methyl carbonate.

As a nonlimiting example, at least one R₁ from Structure (I) of the plurality of groups in the carbazate-functional compound may be a residue of carbonate, such as cyclic carbonate, and contains a same number of hydroxyl groups as the carbonate of which the at least one R₁ is a residue. As a nonlimiting example, for example, a carbonate having no hydroxyl groups used to form R₁ from Structure (I) may result in at least one R₁ having no hydroxyl groups. For example, a carbonate having one hydroxyl groups (e.g., glycerol carbonate) used to form the R₁ from Structure (I) may result in at least one R₁ having one hydroxyl group.

As a nonlimiting example, a composition that includes the carbazate-functional composition (e.g., the composition in which the carbazate functional compound is formed, as distinguished from coating compositions described herein) may be substantially free of residual hydrazine without performing a subsequent purification process, such that the hydrazine content is less than or equal to 0.5%, as measured in the Examples. The carbazate-functional composition may be essentially free of residual hydrazine, such that the hydrazine content is less than or equal to 0.9%, such as less than or equal to 0.1%. As used herein, “essentially free of residual hydrazine” refers to the carbazate-functional composition that has a hydrazine content that is undetectable, from 0.001% to 0.1%, such as 0.01 to 0.9% or 0.01% to 0.5% and can be less than or equal to 0.05% of a composition measured using high performance liquid chromatography (HPLC).

The carbazate-functional compound having Structure (I) may be formed, as nonlimiting examples, by: (i) reacting a carbonate with hydrazine to form an adduct; (ii) reacting the adduct with a ketone and/or aldehyde to form a hydroxyl-functional blocked carbazate; (iii) reacting the hydroxyl-functional blocked carbazate: (1) with an isocyanate-functional compound, (2) with an anhydride-functional compound, (3) with an epoxy-functional compound, (4) with an aminoplast linkage, to form a blocked carbazate-functional compound; and (iv) unblocking the blocked carbazate-functional compound to form a carbazate-functional compound and/or (5) in a Michael addition reaction to form a blocked carbazate-functional compound. The carbazate-functional compound formed according to this process may include a different structure (e.g., at least one R₁ from the plurality of groups of structure (I) being free of a hydroxyl-functional group) and/or include a lower residual hydrazine content compared to carbazate-functional compounds formed by post-reacting a carbonate-functional resin with hydrazine.

At step (i), a carbonate may be reacted with hydrazine to form an adduct. The adduct may be a hydroxyl-functional adduct.

As used herein, “hydrazine” refers to hydrazine (H₂N—NH₂) or a compound having a hydrazine functional group of the formula —NHNH₂. The carbonate may be any of those previously described in connection with R₁. The adduct may include a hydrazine-functional group. The adduct may include a hydroxyl group.

At step (ii), the adduct may be reacted with a ketone and/or aldehyde-functional compound to form a hydroxyl-functional blocked carbazate. The hydroxyl-functional blocked carbazate may include a blocked carbazate-functional group and a hydroxyl functional group.

The ketone and/or aldehyde-functional compound may include a ketone and/or aldehyde functional group reactive with the adduct, such as the hydrazine functional group of the adduct. Non-limiting examples of the ketone-functional compounds include methyl isobutyl ketone (MIBK), methyl ethyl ketone (MEK), acetone, and mixtures or combinations thereof. The ketone-functional compound may include a ketone-containing solvent such that a solution that includes a blocked carbazate compound (formed in this step or any of the blocked carbazates formed during a later-described step) and the ketone-containing solvent is formed, and the blocking group of the blocked carbazate compound may be derived from the ketone-containing solvent. Non-limiting examples of aldehyde-functional compounds include formaldehyde, acetaldehyde, propanal, butanal, and mixtures or combinations thereof. The aldehyde-functional compound may include an aldehyde-containing solvent such that a solution that includes a blocked carbazate compound (formed in this step or any of the blocked carbazates formed during a later-described step) and the aldehyde-containing solvent is formed, and the blocking group of the blocked carbazate compound may be derived from the aldehyde-containing solvent.

The hydroxyl-functional blocked carbazate formed from step (ii) may include the following structure:

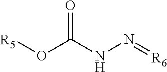

where R₅ includes a compound that includes a hydroxyl group, where R₆ includes a carbon atom-containing group having the carbon atom double bonded to the nitrogen.

At step (iii), the hydroxyl-functional blocked carbazate may be reacted with: (1) with an isocyanate-functional compound [forming at least a portion of a urethane linkage], (2) with an anhydride-functional compound [forming at least a portion of an ester linkage], (3) with an epoxy-functional compound [forming at least a portion of an ether linkage], (4) with an aminoplast linkage [forming at least a portion of an ether linkage], to form a blocked carbazate-functional compound and/or (5) in a Michael addition reaction to form a blocked carbazate-functional compound.

The urethane-functional blocked carbazate formed during step (iii) may include a blocked carbazate-functional group and a linkage X, where X forms at least a portion of a urethane linkage. The urethane linkage may be the result of the hydroxyl group from the hydroxyl-functional blocked carbazate reacting with an isocyanate-functional compound.

Non-limiting examples of suitable isocyanate-functional compounds include isophorone diisocyanate (IPDI), dicyclohexylmethane 4,4′-diisocyanate (H12MDI), cyclohexyl diisocyanate (CHDI), trimethylhexamethylene diisocyanate (TMDI), m-tetramethylxylylene diisocyanate (m-TMXDI), p-tetramethylxylylene diisocyanate (p-TMXDI), ethylene diisocyanate, 1,2-diisocyanatopropane, 1,3-diisocyanatopropane, 1,6-diisocyanatohexane (hexamethylene diisocyanate or HDI), 1,4-butylene diisocyanate, lysine diisocyanate, 1,4-methylene bis-(cyclohexyl isocyanate), toluene diisocyanate (TDI), m-xylylenediisocyanate (MXDI) and p-xylylenediisocyanate, 4-chloro-1,3-phenylene diisocyanate, 1,5-tetrahydro-naphthalene diisocyanate, 4,4′-dibenzyl diisocyanate, 1,2,4-benzene triisocyanate, xylylene diisocyanate (XDI), and prepolymers or mixtures or combinations thereof.

The urethane-functional blocked carbazate formed during step (iii) may include the following structure:

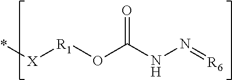

where R₆ includes a carbon atom-containing group having the carbon atom double bonded to the nitrogen.

The ester-functional blocked carbazate formed during step (iii) may include a blocked carbazate-functional group and a linkage X, where X forms at least a portion of an ester linkage. The ester linkage may be the result of the hydroxyl group from the hydroxyl-functional blocked carbazate reacting with an anhydride-functional compound.

Non-limiting examples of suitable anhydride-functional compounds include maleic anhydride, hexahydrophthalic anhydride, methyl hexahydrophthalic anhydride, phthalic anhydride, trimellitic anhydride, succinic anhydride, chlorendic anhydride, alkenyl succinic anhydride, and substituted alkenyl anhydrides such as octenyl succinic anhydride, and derivatives, prepolymers, mixtures and/or combinations thereof.

The ester-functional blocked carbazate formed during step (iii) may include the following structure:

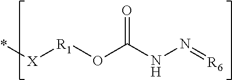

where R₆ includes a carbon atom-containing group having the carbon atom double bonded to the nitrogen.

The ether-functional blocked carbazate formed during step (iii) may include a blocked carbazate-functional group and a linkage X, where X forms at least a portion of an ether linkage. The ether linkage may be the result of the hydroxyl group from the hydroxyl-functional blocked carbazate reacting with an epoxy-functional compound. The ether linkage may be the result of the hydroxyl group from the hydroxyl-functional blocked carbazate reacting with an aminoplast linkage.

Non-limiting examples of suitable epoxy-functional compounds include a cycloaliphatic epoxy; an aliphatic epoxy; a hexahydrophthalic anhydride-based diester epoxy; a cyclohexane dimethanol-based epoxy; a neopentyl glycol-based epoxy; a polyglycidyl ether epoxy (e.g., 1,4-butanediol diglycidyl ether); an aromatic polyfunctional epoxy; a bisphenol-A bisepoxide; a hydrogenated bisphenol-A bisepoxide; a triglycidyl ether of trimethylolpropane; a Novolac (low molecular weight, such as than less 10,000 Daltons, polymers derived from phenols and formaldehyde) epoxy; a bisepoxide of bis(3,5-dimethyl-4-hydroxyphenyl) methane, bis(4-hydroxy-3,5-dimethylphenyl)methanone, and/or 2,2-Bis(3,5-dimethyl-4-hydroxyphenyl)propane; and derivatives, prepolymers, mixtures and/or combinations. Non-limiting examples of suitable compounds that include an aminoplast linkage includes melamine, N-(butoxymethyl) acrylamide, N-(isobutoxymethyl) acrylamide, N-(hydroxymethyl) acrylamide and combinations thereof. The compound that includes the aminoplast linkage may include condensates of amines and/or amides with aldehyde. As a nonlimiting example, the condensate of melamine with formaldehyde is a suitable compound that includes an aminoplast linkage.

The ether-functional blocked carbazate formed during step (iii) may include the following structure:

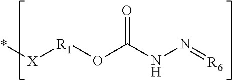

where R₆ includes a carbon atom-containing group having the carbon atom double bonded to the nitrogen.

At step (iv), the blocked carbazate-functional compound (whether urethane, ester, and/or ether functional) may be unblocked to form the carbazate-functional compound, such as the carbazate-functional compound having groups of Structure (I). The blocked carbazate-functional compound may be unblocked by exposing the blocked carbazate-functional compound to conditions sufficient to remove the blocking group to expose/form the carbazate-functional group and, therefore, the carbazate-functional compound. The unblocking conditions may include exposing the blocked carbazate-functional compound in the presence of water to an elevated temperature, at which elevated temperature the blocking groups are removed. The unblocking temperature may be at least 25° C. The term “in the presence of water” means from 3 to 80 wt. %, such as from 5-70 wt. % water based on the weight of a liquid medium. The unblocking temperature may range from 25° C.-150° C., such as from 50° C.-120° C. The unblocking temperature may be up to 150° C. The unblocking conditions may include, without limitation, the inclusion of a catalyst in the aqueous medium, such as an acid catalyst, examples of which include phenyl phosphonic acid, 2-ethylhexyl acid phosphate, dodecyl benzene sulfonic acid, para-toluene sulfonic acid, or a combination thereof. The unblocked carbazate-functional compound may then subsequently react with a functional group on the same or different compound across the exposed carbazate-functional group, such as in a crosslinking reaction.

An ether linkage may be the result of the hydroxyl group from the hydroxyl-functional blocked carbazate reacting in a Michael addition reaction. In the Michael addition reaction, the hydroxyl group may be a nucleophile undergoing an addition reaction with a compound having an activated double bond, such as an acrylate functional material, a maleic ester, a fumaric ester, and/or an itaconic acid ester, to form an ether linkage.

The carbazate functional compound may be rendered water dispersible. As one non-limiting example, the carbazate-functional compound may be prepared using a hydrophilic group, such as a carboxyl group and/or by incorporating polyethylene glycol into a carbazate-functional compound such that it is water dispersible. As a nonlimiting example, a neutralizing amine can be included with the carbazate-functional compound that includes a carboxyl group to at least partially neutralize the acid-functional groups to form a salt. Suitable neutralizing amines include, but are not limited to, ammonium hydroxide, dimethyl amine, trimethylamine, triethylamine, monoethanolamine, diisopropanolamine, diethanolamine, dimethylethanolamine, or a combination thereof.

As a nonlimiting example, the carbazate functional compound can be rendered water dispersible by forming the ammonium salt of the NH₂ group in Structure (I) (as a nonliiting example N⁺H₃ Y⁻) where the counter ion (Y) can include Cl⁻, Br⁻, I⁻, SO₄⁻, PO₄, BF₄⁻, N₂SO₃⁻, anions of carbonic acid, RCOO⁻ and combinations thereof, where R can include one or more of C₁ to C₁₂ aliphatic or aromatic groups. Nonlimiting examples of RCOO⁻ include anions of carboxylic acids such as formic acid, acetic acid, lactic acid, and benzoic acid. The ammonium salt of the carbazate functional compound can be selected and/or neutralized so that its crosslinking capability is not substantially impacted.

The present disclosure is further directed to a (thio)carbazate-functional compound. As used herein, “(thio)carbazate” refers to a thiocarbazate or a carbazate. The carbazate-functional compound encompassed by the term (thio)carbazate is identical to the carbazate-functional compound previously described. Aspects of the thiocarbazate-functional compound different from the carbazate-functional compound will be described hereinafter. The disclosure from the carbazate-functional compound also applies to the thiocarbazate-functional compound described hereinafter except where otherwise indicated.

As used herein, a “thiocarbazate-functional” compound refers to a compound that includes the structure of the carbazate functional group and/or the carbazate linkage previously provided except at least one of the oxygen atoms is replaced with a sulfur atom.

As used herein, a “thiourethane linkage” refers to the structure of the urethane linkage previously provided except at least one of the oxygen atoms is replaced with a sulfur atom.

As used herein, a “thioester linkage” refers to the structure of the ester linkage previously provided except at least one of the oxygen atoms is replaced with a sulfur atom.

As used herein, a “thioether linkage” refers to the structure of the ether linkage previously provided except the oxygen atom is replaced with a sulfur atom.

The (thio)carbazate-functional compound includes a plurality of groups (represented within the brackets [ ]) that include the following structure (II):

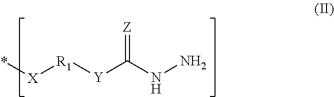

where X forms at least a portion of a (thio)urethane linkage, a (thio)ester linkage, or a (thio)ether linkage, where each Y and Z is independently selected from O and S, where at least one R₁ from the plurality of groups is free of a hydroxyl-functional group. It will be appreciated what when Y and Z are both O, Structure (II) is identical to Structure (I), with the exception that X from Structure (II) may form at least a portion of: a thiourethane linkage, a thioester linkage, or a thioether linkage. For the thiocarbazate-functional compound, at least one of, and potentially both, Y and Z may be S and/or X may form at least a portion of a thiourethane linkage, thioester linkage, and/or thioether linkage.

The present disclosure is also directed to a hydroxyl and/or thiol-functional blocked (thio)carbazate compound including:
a blocked (thio)carbazate-functional group; and a hydroxyl and/or thiol-functional group;
a blocked (thio)carbazate compound including: a blocked (thio)carbazate-functional group;
and a linkage, X, as follows:

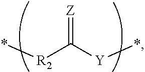

where each Y and Z is independently selected from O and S, and R₂ is NH;
a blocked (thio)carbazate compound including: a blocked (thio)carbazate-functional group; and a linkage, X, as follows:

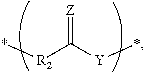

where each Y and Z is independently selected from O and S, and R₂ includes a carbon atom bonded to the (thio) carbonyl carbon and without limitation can include at least one linear or branched alkyl or alkylene group, aryl or arylene group, optionally including one or more functional groups; hydrogen atom; or some combination thereof;
a blocked (thio)carbazate compound including: a blocked (thio)carbazate-functional group; and a linkage, X, as follows:

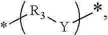

where each Y is independently selected from O and S, where R₃ includes a carbon atom bonded to the Y;
a compound formed by: reacting a (thio) carbonate, such as a cyclic (thio) carbonate, with hydrazine to form an adduct; reacting the adduct with a ketone and/or aldehyde to form a hydroxyl and/or thiol-functional blocked (thio)carbazate; and reacting the hydroxyl and/or thiol-functional blocked (thio)carbazate: (1) with an isocyanate-functional compound, (2) with an anhydride-functional compound, (3) with an epoxy-functional compound, (4) in a Michael addition reaction, and/or (5) with an aminoplast linkage, to form a blocked (thio)carbazate-functional compound.

The X from Structure (II) may form at least a portion of a (thio)urethane linkage. Thus, X may form at least a portion of a (thio)urethane linkage that includes the following structure:

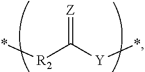

where R₂ is NH.

The X from Structure (II) may form at least a portion of a (thio)ester linkage. Thus, X may form at least a portion of a (thio)ester linkage and includes the following structure:

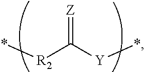

where R₂ includes a carbon atom bonded to the (thio) carbonyl carbon and at least one alkyl group, aryl group, hydrogen atom, or some combination thereof.

The X from Structure (II) may form at least a portion of a (thio)ether linkage. Thus, X may form at least a portion of a (thio)ether linkage and includes the following structure:

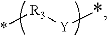

where R₃ includes a carbon atom bonded to the Y. The carbon atom may be part of an alkyl group.

The X from Structure (II) may form at least a portion of a (thio)ether linkage. Thus, X may form at least a portion of a (thio)ether linkage and includes the following structure:

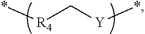

where R₄ includes a carbon atom bonded to the CH₂—Y. The carbon atom may be part of an alkyl group.

From Structure (II), R₁ may be a residue of a (thio) carbonate, such as any of the previously described cyclic carbonates or a cyclic thiocarbonate of any of those carbonates previously described except having at least one oxygen atom replaced with a sulfur atom. Non-limiting examples of a cyclic (thio) carbonate include 1,3-dithiolan-2-one and 1,3-oxathiolane-2-thione. The (thio) carbonate may include a hydroxyl group and/or a thiol group.

The (thio)carbazate-functional compound having Structure (II) may be formed by: (i) reacting a (thio) carbonate with hydrazine to form an adduct; (ii) reacting the adduct with a ketone and/or aldehyde to form a hydroxyl and/or thiol-functional blocked (thio)carbazate; (iii) reacting the hydroxyl and/or thiol-functional blocked (thio)carbazate: (1) with an isocyanate-functional compound, (2) with an anhydride-functional compound, (3) with an epoxy-functional compound, (4) in a Michael addition reaction [where (4) is specific to the thiol-functional group], to form a blocked (thio)carbazate-functional compound, and/or (5) with an aminoplast linkage; and (iv) unblocking the blocked (thio)carbazate-functional compound to form a (thio)carbazate-functional compound. The (thio)carbazate-functional compound formed according to this process may include a different structure (e.g., at least one R₁ from the plurality of groups of structure (II) being free of a hydroxyl-functional group) and/or include a lower residual hydrazine content compared to (thio)carbazate-functional compounds formed by post-reacting an isocyanate-functional resin with hydrazine.

The present disclosure is also directed to a composition including carbazate-functional compound including a plurality of groups including the following structure:

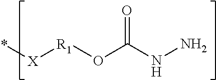

where X forms at least a portion of: a urethane linkage, an ester linkage, or an ether linkage, where the composition is substantially free of residual hydrazine.

The present disclosure is also directed to carbazate-functional compound including a plurality of groups including the following structure:

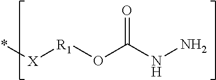

where X forms at least a portion of: a urethane linkage, an ester linkage, or an ether linkage, where at least one R₁ from the plurality of groups is a residue of carbonate, such as cyclic carbonate, and contains a same number of hydroxyl groups as the carbonate of which the at least one R₁ is a residue.

At step (i), a (thio) carbonate may be reacted with hydrazine to form an adduct, and the (thio) carbonate may be as described in connection with the carbonates used to form the adduct in step (i) of forming the carbazate-functional compound having groups of Structure (I) with at least one of the oxygen atoms replaced with a sulfur atom.

At step (ii), the adduct may be reacted with a ketone and/or aldehyde-functional compound to form a hydroxyl and/or thiol-functional blocked (thio)carbazate. The hydroxyl and/or thiol-functional blocked (thio)carbazate may include a blocked (thio)carbazate-functional group and a hydroxyl and/or thiol functional group.

The ketone and/or aldehyde-functional compound may be the same ketone and/or aldehyde-functional compound previously described in connection with step (ii) of forming the carbazate-functional compound having Structure (I). The ketone and/or aldehyde-functional compound may include a ketone and/or aldehyde-containing solvent such that a solution that includes a blocked (thio)carbazate compound (formed in this step or any of the blocked (thio)carbazates formed during a later-described step) and the ketone and/or aldehyde-containing solvent is formed, and the blocking group of the blocked (thio)carbazate compound may be derived from the ketone and/or aldehyde-containing solvent.

The hydroxyl and/or thiol-functional blocked (thio)carbazate formed from step (ii) may include the following structure:

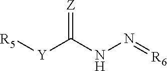

where R₅ includes a compound that includes a hydroxyl and/or thiol group, where R₆ includes a carbon atom-containing group having the carbon atom double bonded to the nitrogen, where each Y and Z is independently selected from O and S.

At step (iii), the hydroxyl and/or thiol-functional blocked (thio)carbazate may be reacted with: (1) with an isocyanate-functional compound [forming at least a portion of a (thio)urethane linkage], (2) with an anhydride-functional compound [forming at least a portion of a (thio)ester linkage], (3) with an epoxy-functional compound [forming at least a portion of a (thio)ether linkage], (4) in a Michael addition reaction [forming at least a portion of a (thio)ether linkage], and/or (5) with an aminoplast linkage [forming at least a portion of a (thio)ether linkage], to form a blocked (thio)carbazate-functional compound.

The (thio)urethane-functional blocked (thio)carbazate formed during step (iii) may include a blocked (thio)carbazate-functional group and a linkage X, where X forms at least a portion of a (thio)urethane linkage. X may include the following structure:

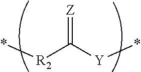

where each Y and Z is independently selected from O and S, and R₂ is NH.

The (thio)urethane linkage may be the result of the hydroxyl and/or thiol group from the hydroxyl and/or thiol-functional blocked (thio)carbazate reacting with an isocyanate-functional compound. The isocyanate-functional compound may be any of those previously described.

The blocked (thio)carbazate compound may include the following structure:

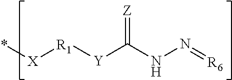

where R₆ includes a carbon atom-containing group having the carbon atom double bonded to the nitrogen

The (thio)ester-functional blocked (thio)carbazate formed during step (iii) may include a blocked (thio)carbazate-functional group and a linkage X, where X forms at least a portion of a (thio)ester linkage. X may include the following structure:

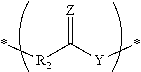

where each Y and Z is independently selected from O and S, and R₂ includes a carbon atom bonded to the (thio) carbonyl carbon and without limitation can include at least one linear or branched alkyl or alkylene group, aryl or arylene group, optionally including one or more functional groups; hydrogen atom; or some combination thereof.

The (thio)ester linkage may be the result of the hydroxyl and/or thiol group from the hydroxyl and/or thiol-functional blocked (thio)carbazate reacting with an anhydride-functional compound. The anhydride-functional compound may be any of those previously described.

The blocked (thio)carbazate compound may include the following structure:

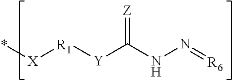

where R₆ includes a carbon atom-containing group having the carbon atom double bonded to the nitrogen.

The (thio)ether-functional blocked (thio)carbazate formed during step (iii) may include a blocked (thio)carbazate-functional group and a linkage X, where X forms at least a portion of a (thio)ether linkage. X may include the following structure:

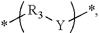

where each Y is independently selected from O and S, where R₃ includes a carbon atom bonded to the Y.

The (thio)ether linkage may be the result of the hydroxyl and/or thiol group from the hydroxyl and/or thiol-functional blocked (thio)carbazate reacting with an epoxy-functional compound. The epoxy-functional compound may be any of those previously described. The (thio)ether linkage may be the result of the hydroxyl and/or thiol group from the hydroxyl and/or thiol-functional blocked carbazate reacting with an aminoplast linkage. The compound that includes an aminoplast linkage may be any of those previously described.

The (thio)ether linkage may be the result of the thiol group from the thiol-functional blocked (thio)carbazate reacting in a Michael addition reaction. In the Michael addition reaction, the thiol group may be a nucleophile undergoing an addition reaction with a compound having an activated double bond, such as an acrylate functional material, a maleic ester, a fumaric ester, and/or an itaconic acid ester, to form a thioether linkage.

The blocked (thio)carbazate compound may include the following structure:

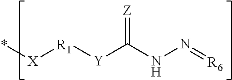

where R₆ includes a carbon atom-containing group having the carbon atom double bonded to the nitrogen.

At step (iv), the blocked (thio)carbazate-functional compound may be unblocked to form the (thio)carbazate-functional compound as described in connection with the carbazate-functional compound.

The disclosure may further include a coating composition that includes the carbazate-functional compound and/or the (thio)carbazate-functional compound. The term “carbazate-functional compound” as used hereinafter in association with inclusion in the coating composition refers to either or both of the carbazate-functional compound and the (thio)carbazate-functional compound.

The coating composition may include at least 1 weight % of the carbazate-functional compound, based on total resin solids, such as at least 5 weight %, at least 10 weight %, at least 20 weight %, at least 30 weight %, at least 40 weight %, at least 50 weight %, at least 60 weight %, or at least 70 weight %. The coating composition may include up to 99 weight % of the carbazate-functional compound, based on total resin solids, such as up to 85 weight %, up to 75 weight %, up to 65 weight %, up to 55 weight %, up to 45 weight %, up to 35 weight %, or up to 25 weight %. The coating composition may include from 1 to 99 weight % of carbazate-functional compound, based on total resin solids, such as from 5 to 85 weight %, from 10 to 75 weight %, from 20 to 65 weight %, or from 30 to 55 weight %.

The carbazate-functional compound may have a carbazate equivalent weight of from 200 to 10,000, such as 200 to 7,500 or 200 to 5,000, calculated based on the number of equivalents of hydroxy functional blocked carbazate.

The coating composition may include an aqueous medium into which the carbazate-functional compound is dispersed (forming an aqueous dispersion) or dissolved (at ambient temperature) to form a solution in the aqueous medium. The carbazate-functional compound may include a carboxyl group or other suitable functional group so as to render the carbazate-functional compound water dispersible.

As used herein, an “aqueous medium” refers to a liquid medium that includes at least 50 weight % water, based on the total weight of the liquid medium, where the liquid medium is defined as water and organic solvents which are liquid at ambient temperature (20-25° C.) and volatile at 110° C. as measured by ASTM D2369-93. As such, it will be appreciated that the liquid medium basis does not include diluents which are liquid at ambient temperature but not volatile at 110° C. as measured by ASTM D2369-93. Such aqueous liquid mediums can for example include at least 60 weight % water, or at least 70 weight % water, or at least 80 weight % water, or at least 90 weight % water, or at least 95 weight % water, or 100 weight % water, based on the total weight of the liquid medium. The solvents that, if present, make up less than 50 weight % of the liquid medium include organic solvents. Non-limiting examples of suitable organic solvents include polar organic solvents, e.g. protic organic solvents such as glycols, glycol ether alcohols, alcohols, volatile ketones, glycol diethers, esters, and diesters. Other non-limiting examples of organic solvents include aromatic and aliphatic hydrocarbons.

As used herein, the term “dispersion” refers to a two-phase system in which one phase includes finely divided particles distributed throughout a second phase, which is a continuous phase. The continuous phase may include the aqueous medium, in which the polymeric particles (e.g., the carbazate-functional compound and/or the other resins or co-resins) are suspended therein. The particles may have an average particle size of from 20 to 2000 nm, such as from 50 to 1000 nm, from 50 to 500 nm, from 50 to 200 nm, from 70 to 150 nm, or from 80 to 150 nm, as determined with a Zetasizer Nano ZS following the instructions in the Zetasizer Nano ZS ZS “Making Size Measurements” and “Software” Manuals.

As a nonlimiting example, the coating composition may include a first compound and a second compound dispersed and/or dissolved in the aqueous medium, where the second compound includes the carbazate-functional compound and is different from the first compound. The first compound may include a macromolecule, oligomer, and/or polymer. A co-solvent may optionally be included to render the second compound water-dispersible or dissolvable in water at ambient conditions. The first compound and/or the second compound may include a carboxyl group or other suitable functional group so as to render the first compound and/or the second compound and/or the reaction product thereof water dispersible at ambient conditions.

The first compound may be reactive with the carbazate-functional group of the carbazate-functional compound such that the first and second compounds may undergo a crosslinking reaction under sufficient conditions. The coating composition including the first compound and the carbazate-functional compound may be a thermoset coating composition. The term “thermoset” refers to a composition that “sets” upon curing or crosslinking, where the polymer chains of the resins are joined together by covalent bonds. Once cured or crosslinked, a thermosetting resin will not melt upon the application of heat and is insoluble in solvents. The term “thermoplastic” refers to resins where the polymer chains are not joined together by covalent bonds and, thereby, can undergo liquid flow upon heating and can be soluble in certain solvents.

To be reactive with the carbazate-functional group of the carbazate-functional compound, the first compound may include a functional group reactive with the carbazate-functional group, non-limiting examples of which include a ketone-functional group, an aldehyde-functional group, an isocyanate-functional group, an epoxy-functional, a double bond-functional group, and/or a mixture or combination thereof. For example, the first compound may include a ketone-functional group reactive with the carbazate-functional group of the carbazate-functional compound.

As a nonlimiting example, the coating composition may include a self-crosslinkable molecule or particle. As used herein, “self-crosslinkable” refers to a molecule or particle, which can be a dispersed particle or macromolecule, oligomer, and/or polymer, that includes at least one carbazate-functional group and at least one second functional group, reactive with a carbazate-functional group. A co-solvent may optionally be included to render the self-crosslinkable molecule or particle water-dispersible or dissolvable in water. The self-crosslinkable molecule or particle may include a carboxyl group or other suitable functional group so as to render the self-crosslinkable molecule and/or the reaction product thereof water dispersible. The coating composition including the self-crosslinkable molecule or particle may be a thermoset coating composition. As non-limiting examples, the second functional group, reactive with a carbazate-functional group, can include a ketone-functional group, an aldehyde-functional group, an isocyanate-functional group, an epoxy-functional, a double bond-functional group, and/or a mixture or combination thereof.

As a nonlimiting example, the coating composition may include a self-crosslinkable molecule or particle as described herein. Additionally and without limitation, the coating composition may include a combination of (a) one or both of a first compound and a second compound dispersed and/or dissolved in the aqueous medium, where the second compound includes the carbazate-functional compound and is different from the first compound, as described herein and (b) a self-crosslinkable molecule or particle as described herein.

The coating composition may include at least 1 weight % of the first compound, based on total resin solids, such as at least 5 weight %, at least 15 weight % at least 25 weight %, at least 35 weight %, at least 45 weight %, at least 55 weight %, at least 65 weight %, at least 75 weight %, or at least 85 weight %. The coating composition may include up to 99 weight % of the first compound, based on total resin solids, such as up to 95 weight %, up to 85 weight %, up to 75 weight %, up to 65 weight %, up to 55 weight %, up to 45 weight %, up to 35 weight %, or up to 25 weight %. The coating composition may include from 1 to 99 weight % of first compound, based on total resin solids, such as from 5 to 85 weight %, from 15 to 95 weight %, from 25 to 85 weight %, from 35 to 75 weight %, or from 45 to 65 weight %.

The first compound may have a ketone and/or aldehyde equivalent weight of from 150 to 10,000, calculated based on the number of equivalents of ketone and/or aldehyde-functional compound.

The coating composition may include an equivalent ratio of carbazate functional groups: ketone and/or aldehyde functional groups of from 25% to 200%, such as from 75% to 150% or from 90% to 110%.

The first compound and the carbazate functional compound may include the same compound so as to form self-crosslinkable compound, such as a compound having carbazate-functional groups and ketone and/or aldehyde-functional groups.

The first compound and the second compound may react together to form a reaction product having a core-shell structure which includes carbazate and ketone and/or aldehyde functionality. The first compound may include an acrylic compound which is a precursor of an acrylic core formed from acrylic monomers which, when reacted with the second compound, forms the acrylic core of the core-shell structure. The second compound may include the carbazate-functional compound which is a precursor of a carbazate-functional shell which, when reacted with the first compound, forms the carbazate-functional shell of the core-shell structure. When the first compound and second compound are reacted together, the shell may at least partially encapsulate the core to form core-shell structure. The core-shell structure may be formed by reacting the precursor of the carbazate-functional shell with the acrylic monomer from the precursor of the acrylic core. The core and shell may be covalently bonded together, such as by grafting the precursor of the shell to the precursor of the core across an unsaturated double bond of the core through free radical polymerization. The shell may include a carboxyl group or other suitable functional group so as to render the core-shell structure water dispersible.

The core-shell particle may include carbazate functionality on the core and/or the shell and/or may have ketone and/or aldehyde functionality on the core and/or the shell. The core-shell particle may include carbazate and ketone and/or aldehyde functionality on the shell. The core-shell particle may include carbazate and ketone and/or aldehyde functionality on the core. The core-shell particle may include carbazate functionality on the shell and ketone and/or aldehyde functionality on the core. The core-shell particle may include ketone and/or aldehyde functionality on the shell and carbazate functionality on the core. The inclusion of carbazate and ketone and/or aldehyde functionality on the core-shell particles (e.g., the core and/or the shell thereof) may render the core-shell particle self-crosslinkable.

The coating composition may include a core-shell particle that includes carbazate functionality on the core, the shell, or both the core and the shell. The core-shell particle that includes carbazate functionality may be non-self-crosslinkable such as being free of ketone and/or aldehyde-functional groups. Such compound may be considered the carbazate-functional compound.

The coating composition may further include a polyester polymer. The polyester polymer may be obtained from components that includes polytetrahydrofuran and a carboxylic acid or anhydride thereof. The polyester polymer may include a hydroxyl functional group.

The carboxylic acid or anhydride used to form the polyester polymer can be selected from various types of polycarboxylic acids or the anhydrides thereof, such as from a dicarboxylic acid or anhydride thereof, or from a polycarboxylic acid having three or more carboxylic acid groups or the anhydrides thereof. The carboxylic acid or anhydride thereof can also be selected from compounds having aromatic rings or aliphatic structures. As used herein, an “aromatic group” refers to a cyclically conjugated hydrocarbon with a stability (due to delocalization) that is significantly greater than that of a hypothetical localized structure. Further, the term “aliphatic” refers to non-aromatic straight, branched, or cyclic hydrocarbon structures that contain saturated carbon bonds.

Non-limiting examples of carboxylic acids used to form the polyester polymer include, but are not limited to, glutaric acid, succinic acid, malonic acid, oxalic acid, trimellitic acid, phthalic acid, isophthalic acid, hexahydrophthalic acid, adipic acid, maleic acid, anhydrides thereof, or mixtures thereof. Further, suitable acid containing diols include, but are not limited to, 2,2-bis(hydroxymethyl) propionic acid which is also referred to as dimethylolpropionic acid (DMPA), 2,2-bis(hydroxymethyl) butyric acid which is also referred to as dimethylol butanoic acid (DMBA), diphenolic acid, or a combination thereof. As indicated, an anhydride can be used, such as an anhydride of any of the previously described carboxylic acids. The carboxylic acid or anhydride may include trimellitic acid and/or anhydride. Non-limiting examples of such anhydrides include trimellitic anhydride, phthalic anhydride, maleic anhydride, succinic anhydride, malonic anhydride, oxalic anhydride, hexahydrophthalic anhydride, adipic anhydride, and combinations thereof.

As indicated, the carboxylic acid or anhydride thereof can be selected from compounds having aromatic rings or aliphatic structures. For instance, the carboxylic acid or anhydride thereof can be selected from an aromatic compound in which the carboxylic acid or anhydride functional groups are bonded directly to the aromatic ring(s) such that there is no interrupting atoms between the aromatic ring(s) and the attached carboxylic acid or anhydride functional groups (a non-limiting example being trimellitic anhydride).

The polyester polymer can also be prepared with other components in addition to the previously described polytetrahydrofuran and carboxylic acid or anhydride thereof. Non-limiting examples of additional components that can be used to form the polyester polymer include polyols in addition to the polytetrahydrofuran, additional compounds containing one or more carboxylic acid groups or anhydrides thereof, ethylenically unsaturated compounds, polyisocyanates, and combinations thereof.

Non-limiting examples of polyols used to form the polyester polymer include glycols, polyether polyols, polyester polyols, copolymers thereof, and combinations thereof. Non-limiting examples of glycols include ethylene glycol, diethylene glycol, triethylene glycol, 1,2-propylene glycol, 1,3-butylene glycol, tetramethylene glycol, hexamethylene glycol, and combinations thereof, as well as other compounds that include two or more hydroxyl groups and combinations of any of the foregoing. Non-limiting examples of suitable polyether polyols in addition to the polytetrahydrofuran include polyethylene glycol, polypropylene glycol, polybutylene glycol, and combinations thereof. Suitable polyester polyols include those prepared from a polyol comprising an ether moiety and a carboxylic acid or anhydride. Other suitable polyols include, but are not limited to, 1,6-hexanediol, cyclohexanedimethanol, 2-ethyl-1,6-hexanediol, 1,4-butanediol, ethylene glycol, propylene glycol, 1,3-propanediol, 1,4-butanediol, neopentyl glycol, trimethylol propane, 1,2,6-hexantriol, glycerol, or a combination thereof. It is appreciated that the polyol can be selected from diols and/or from compounds having 3 or more hydroxyl groups.

The additional compounds containing one or more carboxylic acid groups or anhydrides that can used to form the polyester polymer include any of the previously described carboxylic acids and anhydrides provided that the additional compound is different from the first carboxylic acid or anhydride. For instance, the components that form the polyester polymer can include both trimellitic anhydride and maleic anhydride.

Non-limiting examples of ethylenically unsaturated monomers, including those containing an acid group, used to form the polyester polymer include (meth)acrylate groups, vinyl groups, or a combination thereof. As used herein, the term “(meth)acrylate” refers to both the methacrylate and the acrylate. Suitable ethylenically unsaturated monomers include, but are not limited to, alkyl esters of (meth)acrylic acid, hydroxyalkyl esters of (meth)acrylic acid, acid group containing unsaturated monomers, vinyl aromatic monomers, or a combination thereof.

Non-limiting examples of suitable alkyl esters of (meth)acrylic acid include methyl (meth)acrylate, ethyl (meth)acrylate, butyl (meth)acrylate, isobutyl (meth)acrylate, ethylhexyl (meth)acrylate, lauryl (meth)acrylate, octyl (meth)acrylate, glycidyl (meth)acrylate, isononyl (meth)acrylate, isodecyl (meth)acrylate, vinyl (meth)acrylate, acetoacetoxyethyl (meth)acrylate, acetoacetoxypropyl (meth)acrylate, or a combination thereof. Other suitable alkyl esters include, but are not limited to, di(meth)acrylate alkyl diesters formed from the condensation of two equivalents of (meth)acrylic acid such as ethylene glycol di(meth)acrylate. Di(meth)acrylate alkyl diesters formed from C₂₋₂₄ diols such as butane diol and hexane diol can also be used.

Non-limiting examples of suitable hydroxyalkyl esters of (meth)acrylic acid include hydroxymethyl (meth)acrylate, hydroxyethyl (meth)acrylate, hydroxypropyl (meth)acrylate, hydroxybutyl (meth)acrylate, or a combination thereof. Suitable acid group containing unsaturated monomers include (meth)acrylic acid, itaconic acid, maleic acid, fumaric acid, crotonic acid, aspartic acid, malic acid, mercaptosuccinic acid, or a combination thereof.

Non-limiting examples of vinyl aromatic monomers used to form the polyester polymer include styrene, 2,4-dimethylstyrene, ethylstyrene, isopropylstyrene, butylstyrene, vinyl naphthalene, vinyl toluene, divinyl aromatic monomers such as divinyl benzene, or a combination thereof.

Non-limiting examples of suitable polyisocyanates used to form the polyester polymer include any of those previously listed.

It is appreciated that the previously described optional additional components can be used to modify or adjust the properties of the polyester polymer and the final coating formed therewith. For instance, the polyester polymer can be formed with additional components, such as an additional polyol, that can provide a faster cure, such as 30 minutes or less, at lower bake temperatures such as temperatures of 80° C. or lower.

The polytetrahydrofuran used to form the polyester polymer can include at least 20 weight % of the components that form the polyester polymer, or at least 30 weight % of the components that form the polyester polymer, or at least 40 weight % of the components that form the polyester polymer. The polytetrahydrofuran can also include up to 50 weight % of the components that form the polyester polymer, or up to 60 weight % of the components that form the polyester polymer, or up to 70 weight % of the components that form the polyester polymer, or up to 80 weight % of the components that form the polyester polymer, or up to 90 weight % of the components that form the polyester polymer. The polytetrahydrofuran can further include an amount within a range such as from 20 weight % to 90 weight % of the components that form the polyester polymer, or from 40 weight % to 80 weight % of the components that form the polyester polymer, or from 50 weight % to 70 weight % of the components that form the polyester polymer, or from 30 weight % to 40 weight % of the components that form the polyester polymer.

The carboxylic acid or anhydride used to form the polyester polymer can include at least 5 weight % of the components that form the polyester polymer, or at least 8 weight % of the components that form the polyester polymer. The carboxylic acid or anhydride can also include up to 20 weight % of the components that form the polyester polymer, or up to 15 weight % of the components that form the polyester polymer, or up to 12 weight % of the components that form the polyester polymer. The carboxylic acid or anhydride can further include a range of from 5 weight % to 20 weight % of the components that form the polyester polymer, or from 8 weight % to 15 weight % of the components that form the polyester polymer, or from 8 weight % to 12 weight % of the components that form the polyester polymer, or from 7 weight % to 10 weight % of the components that form the polyester polymer.

It is appreciated that one or more of the previously described additional components can make up the remaining amount of components used to form the polyester polymer. For example, the polyester polymer can be prepared with polytetrahydrofuran, a carboxylic acid or anhydride, a polyol that is different from the polytetrahydrofuran, and another carboxylic acid or anhydride that is different from the first carboxylic acid or anhydride.

The resulting polyester polymer prepared from the previously described components may include ether linkages and/or carboxylic acid functional groups. The polyester polymer can also include urethane linkages as well as additional functional groups, such as hydroxyl functional groups. For instance, the polyester polymer can include ether linkages, ester linkages, carboxylic acid functional groups, and hydroxyl functional groups. The polyester polymer can also include additional linkages and functional groups including, but not limited to, the previously described additional functional groups.

The polyester polymer can have an acid value of at least 15, at least 20, at least 30, at least 35, or at least 40, based on the total resin solids of the polyester polymer. The polyester polymer can have an acid value of up to 60, up to 55, up to 50, up to 45, up to 40, up to 35, or up to 30, based on the total resin solids of the polyester polymer. The polyester polymer can have an acid value ranging from 15 to 60, such as from 20 to 30, from 20 to 50, from 20 to 60, from 30 to 50, from 30 to 60, from 35 to 60, from 35 to 50, from 40 to 50, or from 40 to 60, based on the total resin solids of the polyester polymer. Any acid value or hydroxyl value recited herein is determined using a Metrohm 798 MPT Titrino automatic titrator, manufactured by Metrohm AG (Herisau, Switzerland), according to ASTM D 4662-15 and ASTM E 1899-16, respectively.

The acid functionality of the polyester polymer can have a pKa of less than 5, or less than 4, or less than 3.5, or less than 3, or less than 2.5, or less than 2. The acid functionality of the polyester polymer can be within a pKa range such as for example from 1.5 to 4.5. The pKa value is the negative (decadic) logarithms of the acidic dissociation constant, and is determined according to the titration method described in Lange's Handbook of Chemistry, 15th edition, section 8.2.1.

The carboxylic acid functionality found on the polyester polymer can be provided by the first carboxylic acid or anhydride only. Alternatively, when additional carboxylic acid functional compounds and/or anhydrides are used to form the polymer, the carboxylic acid functionality found on the polymer is provided by the first carboxylic acid or anhydride and the additional carboxylic acid functional compounds and/or anhydrides.

The polyester polymer can also include a hydroxyl equivalent weight of from 250 to 5000, such as from 1500 to 5000 or from 2000 to 3000, as measured by reacting the dried polyester polymer with an excess amount of acetic anhydride and titrating with potassium hydroxide.

The coating composition may include from 5 to 50 weight % of the polyester polymer based on total resin solids of the coating composition, such as from 5 to 40 weight %, from 5 to 30 weight %, from 5 to 20 weight %, from 10 to 40 weight %, from 10 to 30 weight %, or from 10 to 20 weight %.

The coating composition may further include a polymer reactive with the first compound and/or the second compound. The polymer may be obtained from components that include N-(hydroxymethyl) acrylamide, N-(isobutoxymethyl) acrylamide, or a combination thereof.

In addition, the coating composition can include additional materials including, but not limited to, optional additional resins such as additional film-forming resins. The additional resin can include any of a variety of thermoplastic and/or thermosetting film-forming resins known in the art. Non-limiting examples of suitable additional resins include polyurethanes, polyesters, and/or polyethers other than those previously described, polyamides, polysiloxanes, fluoropolymers, polysulfides, polythioethers, polyureas, (meth)acrylic resins (e.g., acrylic dispersions), epoxy resins, vinyl resins, copolymers thereof, or mixtures thereof. The additional resin may include a core-shell particle different from those previously described. The additional resin may include a non-core-shell particle resin. The additional resin may include a grind resin used to introduce pigment into the coating composition.

The additional resin can have any of a variety of reactive functional groups including, but not limited to, carboxylic acid groups, amine groups, epoxide groups, hydroxyl groups, thiol groups, carbamate groups, amide groups, urea groups, isocyanate groups (including blocked isocyanate groups), (meth)acrylate groups, and combinations thereof. Thermosetting coating compositions typically include a crosslinker that may be selected from any of the crosslinkers known in the art to react with the functionality of the resins used in the coating compositions. Alternatively, a thermosetting film-forming resin can be used having functional groups that are reactive with themselves; in this manner, such thermosetting resins are self-crosslinking.

The coating composition may include the optional additional resin. When the optional additional resin is included in the coating composition, the coating composition may include from 5 to 40 weight % of the additional resin based on total resin solids, such as from 5 to 30 weight %, from 5 to 20 weight %, from 10 to 40 weight %, from 10 to 30 weight %, from 20 to 30 weight %, or from 15 to 30 weight %. The coating composition may include up to 40 weight % of the additional resin based on total resin solids, such as up to 30 weight %, up to 20 weight %, or up to 10 weight %.

The coating composition may include an acid catalyst. The acid catalyst may be a separate component from the first and second compounds, such as a phosphoric or phosphonic or sulfonic acid catalyst. Non-limiting examples include phenyl phosphonic acid, 2-ethylhexyl acid phosphate, dodecyl benzene sulfonic acid, para-toluene sulfonic acid, or a combination thereof. The separate acid catalyst component may include a separate polymer (different from the first and second compounds) that includes the acid catalyst, such as an acrylic polymer that includes an acid catalyst or an epoxy resin that includes an acid catalyst (e.g., a phosphatized acrylic or phosphatized epoxy resin). The acid catalyst may be bonded to the first and/or second compound, such as carboxylic acid. For example, the first and/or second compound may include a phosphonic and/or sulfonic acid acrylate, such as a phosphonic and/or sulfonic acid acrylate of the above-described core-shell particles.

The acid catalyst may include carboxylic acid functional groups formed on the first and/or second compound. The carboxylic acid functional groups may be obtained from a carboxylic acid or anhydride thereof having a pKa of less than 5.5, such as dimethylolpropionic acid (DMPA). The carboxylic acid functional groups may be obtained from a carboxylic acid or anhydride thereof having a pKa of less than 3, such as trimellitic anhydride.

The coating composition may be substantially free (less than 5 weight % based on total resin solids) of unreacted polyisocyanate. The coating composition may be essentially free (less than 1 weight % based on total resin solids) of unreacted polyisocyanate. The coating composition may be free (0 weight % based on total resin solids) of unreacted polyisocyanate. As used herein, “unreacted isocyanate” refers to a molecule having at least one —N═C—O group at ambient temperature.

The coating composition may include an adhesion promoter. The adhesion promotor may include a silane compound. The adhesion promotor may be reactive with the substrate to which the coating composition is applied and the resin of the coating composition so as to enhance adhesion of the cured coating to the substrate.

The coating composition may further include a crosslinker reactive with functional groups and/or linkages on: (i) the first compound; (ii) second compound; and/or (iii) a reaction product obtained from the first and second compounds. The crosslinker may include an isocyanate (for 2K systems), a blocked isocyanate, a carbodiimide (for 1K systems or 2K systems), an aminoplast, an oxazoline, an alpha effect based nucleophile, hydrazine, hydrazide or combinations thereof. The aminoplast crosslinker may include melamine. The aminoplast crosslinker may include condensates of amines and/or amides with aldehyde. For example, the condensate of melamine with formaldehyde is an example of a suitable aminoplast.

As used herein, the term “alpha effect based nucleophile” refers to a nucleophile having increased nucleophilicity of an atom due to the presence of an adjacent (alpha) atom having a lone pair of electrons. Non-limiting examples of alpha effect based nucleophiles include semi-carbazide functional groups and/or linkages, carbazate functional groups and/or linkages, oxime functional groups, and aminoxy functional groups and/or linkages. Nonlimiting examples of suitable alpha effect based nucleophiles are disclosed in published international application WO2022125887A1 at paragraphs [0021] through [0023], including Table A, the specifically cited portions of which are incorporated herein by reference.

The coating composition may be a one-component (1K) curing composition. As used herein, a “1K curing composition” refers to a composition where all the coating components are maintained in the same container after manufacture, during storage, and the like, and may remain stable for longer than 1 month at conditions of 40-120° F. (4-49° C.) at 0-95% relative humidity, such as longer than 3 months, longer than 6 months, longer than 9 months, or longer than 12 months. A 1K curing composition can be applied to a substrate and cured by any conventional means, such as by heating, forced air, and the like.

The coating composition may be a multi-component composition, such as a two component composition (“2K”) or more, which has at least two components that are maintained in a different container after manufacture, during storage, etc. prior to application and formation of the coating over a substrate.

The coating composition can also include additional materials such as a pigment. The pigment may include a finely divided solid powder that is insoluble (at ambient conditions), but wettable, under the conditions of use. A pigment can be organic or inorganic and can be agglomerated or non-agglomerated. Pigments can be incorporated into the coating by use of a grind vehicle, such as an acrylic grind vehicle, the use of which will be familiar to one skilled in the art. The first compound, second compound, and/or polyester polymer may function as the grind vehicle for the pigment.

Suitable pigments and/or pigment compositions include, but are not limited to, carbazole dioxazine crude pigment, azo, monoazo, diazo, naphthol AS, salt type (flakes), benzimidazolone, isoindolinone, isoindoline and polycyclic phthalocyanine, quinacridone, perylene, perinone, diketopyrrolo pyrrole, thioindigo, anthraquinone, indanthrone, anthrapyrimidine, flavanthrone, pyranthrone, anthanthrone, dioxazine, triarylcarbonium, quinophthalone pigments, diketo pyrrolo pyrrole red (“DPPBO red”), titanium dioxide, carbon black, or mixtures thereof.

The pigment used with the coating composition can also include a special effect pigment. As used herein, a “special effect pigment” refers to a pigment that interacts with visible light to provide an appearance effect other than, or in addition to, a continuous unchanging color. Suitable special effect pigments include those that produce one or more appearance effects such as reflectance, pearlescence, metallic sheen, texture, phosphorescence, fluorescence, photochromism, photosensitivity, thermochromism, goniochromism, and/or color-change, such as transparent coated mica and/or synthetic mica, coated silica, coated alumina, aluminum flakes, a transparent liquid crystal pigment, a liquid crystal coating, or a combination thereof.

In some examples, the coating composition may be a clearcoat substantially free of a pigment. Substantially free of a pigment may mean that the coating composition includes less than 3 weight % of pigment, based on the solids of the coating composition, such as less than 2 weight %, less than 1 weight %, or 0 weight %.

Other suitable materials that can be used with the coating composition include, but are not limited to, plasticizers, abrasion resistant particles, anti-oxidants, hindered amine light stabilizers, UV light absorbers and stabilizers, surfactants, flow and surface control agents, thixotropic agents, catalysts, reaction inhibitors, and other customary auxiliaries.

The coating composition may be curable at a temperature of less than or equal to 100° C., such that, when the coating composition is applied to a substrate to form a layer having a thickness from 5 to 100 microns and baked at 100° C. for 30 minutes, the layer achieves at least 35, such as at least 45, or at least 50 MEK double rubs as measured according to the Solvent Resistance Test described herein. The coating composition may be curable at a temperature of less than or equal to 80° C., such that, when the coating composition is applied to a substrate to form a layer having a thickness from 5 to 100 microns and baked at 80° C. for 30 minutes, the layer achieves at least 35, such as at least 45, or at least 50 MEK double rubs as measured according to the Solvent Resistance Test described herein.

The coating composition may be applied to a substrate and cured to form a coating over at least a portion of the substrate.

The substrate over which the coating composition may be applied includes a wide range of substrates. For example, the coating composition of the present disclosure can be applied to a vehicle substrate, an industrial substrate, an aerospace substrate, and the like.

The vehicle substrate may include a component of a vehicle. In the present disclosure, the term “vehicle” is used in its broadest sense and includes all types of aircraft, spacecraft, watercraft, and ground vehicles. For example, the vehicle can include, but is not limited to an aerospace substrate (a component of an aerospace vehicle, such as an aircraft such as, for example, airplanes (e.g., private airplanes, and small, medium, or large commercial passenger, freight, and military airplanes), helicopters (e.g., private, commercial, and military helicopters), aerospace vehicles (e.g., rockets and other spacecraft), and the like). The vehicle can also include a ground vehicle such as, for example, animal trailers (e.g., horse trailers), all-terrain vehicles (ATVs), cars, trucks, buses, vans, heavy duty equipment, tractors, golf carts, motorcycles, bicycles, snowmobiles, trains, railroad cars, and the like. The vehicle can also include watercraft such as, for example, ships, boats, hovercrafts, and the like. The vehicle substrate may include a component of the body of the vehicle, such as an automotive hood, door, trunk, roof, and the like; such as an aircraft or spacecraft wing, fuselage, and the like; such as a watercraft hull, and the like.

The coating composition may be applied over an industrial substrate which may include tools, heavy duty equipment, furniture such as office furniture (e.g., office chairs, desks, filing cabinets, and the like), appliances such as refrigerators, ovens and ranges, dishwashers, microwaves, washing machines, dryers, small appliances (e.g., coffee makers, slow cookers, pressure cookers, blenders, etc.), metallic hardware, extruded metal such as extruded aluminum used in window framing, other indoor and outdoor metallic building materials, and the like.

The coating composition may be applied over storage tanks, windmills, nuclear plant components, packaging substrates, wood flooring and furniture, apparel, electronics, including housings and circuit boards, glass and transparencies, sports equipment, including golf balls, stadiums, buildings, bridges, and the like.

The substrate can be metallic or non-metallic. Metallic substrates include, but are not limited to, tin, steel (including electrogalvanized steel, cold rolled steel, hot-dipped galvanized steel, among others), aluminum, aluminum alloys, zinc-aluminum alloys, steel coated with a zinc-aluminum alloy, and aluminum plated steel. Non-metallic substrates include polymeric materials, plastic and/or composite material, polyester, polyolefin, polyamide, cellulosic, polystyrene, polyacrylic, poly(ethylene naphthalate), polypropylene, polyethylene, nylon, ethylene vinyl alcohol (EVOH), polylactic acid, other “green” polymeric substrates, poly(ethyleneterephthalate) (PET), polycarbonate, polycarbonate and acrylonitrile butadiene styrene copolymer (PC/ABS), wood, veneer, wood composite, particle board, medium density fiberboard, cement, stone, glass, paper, cardboard, textiles, leather, both synthetic and natural, and the like. The substrate may include a metal, a plastic and/or composite material, and/or a fibrous material. The fibrous material may include a nylon and/or a thermoplastic polyolefin material with continuous strands or chopped carbon fiber. The substrate can be one that has already been treated in some manner, such as to impart visual and/or color effect, a protective pretreatment or other coating layer, and the like.

The coating composition of the present disclosure may be particularly beneficial when applied to a metallic substrate. The coatings of the present disclosure may be particularly beneficial when applied to metallic substrates that are used to fabricate automotive vehicles, such as cars, trucks, and tractors.

The coating composition may be applied to a substrate having multiple components, where the coating composition is simultaneously applied to the multiple components and simultaneously cured to form a coating over the multiple components without deforming, distorting, or otherwise degrading any of the components. The components may be parts of a larger whole of the substrate. The components may be separately formed and subsequently arranged together to form the substrate. The components may be integrally formed to form the substrate.

Non-limiting examples of components of a substrate in the vehicle context include a vehicle body (e.g., made of metal) and a vehicle bumper (e.g., made or plastic) which are separately formed and subsequently arranged to form the substrate of the vehicle. Further examples include a plastic automotive component, such as a bumper or fascia in which the bumper or fascia includes regions or subcomponents which include more than one type of substrate. Further examples include aerospace or industrial components that include more than one substrate type. It will be appreciated that other such other multi-component substrates are contemplated within the context of this disclosure.

The multiple components may include at least a first component and a second component, and the first component and the second component may be formed from different materials. As used herein, “different materials” refers to the materials used to form the first and second component having different chemical make-ups.

The different materials may be from the same or different class of materials. As used herein, a “class of materials” refers to materials that may have a different specific chemical make-up but share the same or similar physical or chemical properties. For example, metals, polymers, ceramics, and composites may be defined as different classes of materials. However, other classes of materials may be defined depending on similarities in physical or chemical properties, such as nanomaterials, biomaterials, semiconductors, and the like. Classes of materials may include crystalline, semi-crystalline, and amorphous materials. Classes of materials, such as for polymers, may include thermosets, thermoplastics, elastomers, and the like. Classes of materials, such as for metals, may include alloys and non-alloys. As will be appreciated from the above exemplary list of classes, other relevant classes of materials may be defined based on a given physical or chemical property of materials.

The first component may be formed from a metal, and the second component may be formed from a plastic or a composite. The first component may be formed from a plastic, and the second component may be formed from a metal or a composite. The first component may be formed from a composite, and the second component may be formed from a plastic or a metal. The first component may be formed from a first metal, and the second component may be formed from a second metal different from the first metal. The first component may be formed from a first plastic, and the second component may be formed from a second plastic different from the first plastic. The first component may be formed from a first composite, and the second component may be formed from a second composite different from the first composite. As will be appreciated from these non-limiting examples, any combination of different materials from the same or different classes may form the first and second components.

Examples of combinations of materials include thermoplastic polyolefins (TPO) and metal, TPO and acrylonitrile butadiene styrene (ABS), TPO and acrylonitrile butadiene styrene/polycarbonate blend (ABS/PC), polypropylene and TPO, TPO and a fiber reinforced composite, and other combinations. Further examples include aerospace substrates or industrial substrates that include various components made of a plurality of materials, such as various metal-plastic, metal-composite, and/or plastic-composite containing components. The metals may include ferrous metals and/or non-ferrous metals. Non-limiting examples of non-ferrous metals include aluminum, copper, magnesium, zinc, and the like, and alloys including at least one of these metals. Non-limiting examples of ferrous metals include iron, steel, and alloys thereof.

The first component and the second component (the materials thereof) may exhibit different physical or chemical properties when exposed to elevated temperatures, such as greater than 80° C. to 120° C. For example, the first component may deform, distort, or otherwise degrade at a temperature lower than the second component. Non-limiting examples of material properties which may indicate whether a first component deforms, distorts, or otherwise degrades at a temperature lower than the second component include: heat deflection temperature, embrittlement temperature, softening point, and other relevant material properties associated with deformation, distortion, or degradation of materials.

For example, the first component may deform, distort, or otherwise degrade at temperatures ranging from above 80° C. to 120° C., whereas the second component may not deform, distort, or otherwise degrade at temperatures within or below this range.

When the coating composition is applied to the substrate having multiple components simultaneously, the applied coating composition may be cured at a temperature which does not deform, distort, or otherwise degrade either of the first and second component (the materials thereof). Thus, the curing temperature may be below the temperature at which either of the first component or the second component would deform, distort, or otherwise degrade. The coating composition may be cured at temperatures ranging from 80° C. to 120° C. where neither the first component nor the second component would deform, distort, or otherwise degrade within that range. The coating composition may be cured at temperatures less than or equal to 120° C., less than or equal to 110° C., less than or equal to 100° C., less than or equal to 90° C., or less than or equal to 80° C. where neither the first component nor the second component would deform, distort, or otherwise degrade within these ranges.

Therefore, the coating composition may be curable at relatively low temperatures, within the ranges mentioned above, such that components formed from different materials may be simultaneously coated with the coating composition and cured to form a coating thereover without deforming, distorting, or otherwise degrading either component.

The coating composition may be applied to the substrate by any suitable means, such as spraying, electrostatic spraying, dipping, rolling, brushing, and the like.

The coating composition formed from the coating system can be applied to a substrate to form a pigmented topcoat. The pigmented topcoat may be the topmost coating layer so as not to include a clearcoat or any other coating layer thereover. The pigmented topcoat may be applied directly to the substrate. The pigmented topcoat may be applied over a primer layer or a pretreatment layer.

The coating composition can be applied to a substrate as a coating layer of a multi-layer coating system, such that one or more additional coating layers are formed below and/or above the coating formed from the coating composition.

The coating composition can be applied to a substrate as a primer coating layer of the multi-layer coating system. A “primer coating layer” refers to an undercoating that may be deposited onto a substrate (e.g., directly or over a pre-treatment layer) in order to prepare the surface for application of a protective or decorative coating system.

The coating composition can be applied to a substrate as a basecoat layer of the multi-layer coating system. A “basecoat” refers to a coating that is deposited onto a primer overlying a substrate and/or directly onto a substrate, optionally including components (such as pigments) that impact the color and/or provide other visual impact. A clearcoat may be applied over the basecoat layer.

The coating composition can be applied to a substrate as a topcoat layer of the multi-layer coating system. A “topcoat” refers to an uppermost coating that is deposited over another coating layer, such as a basecoat, to provide a protective and/or decorative layer, such as the previously described pigmented topcoat.

The topcoat layer used with the multi-layer coating system of the present disclosure may be a clearcoat layer, such as a clearcoat layer applied over a basecoat layer. As used herein, a “clearcoat” refers to a coating layer that is at least substantially transparent or fully transparent. The term “substantially transparent” refers to a coating, where a surface beyond the coating is at least partially visible to the naked eye when viewed through the coating. The term “fully transparent” refers to a coating, where a surface beyond the coating is completely visible to the naked eye when viewed through the coating. It is appreciated that the clearcoat can include colorants, such as pigments, provided that the colorants do not interfere with the desired transparency of the clearcoat. The clearcoat can be substantially free or free of pigments.

The coating composition may be applied over a substrate as a layer in a multi-layer coating system. In the multi-layer coating system, a first basecoat layer may be applied over at least a portion of a substrate, where the first basecoat layer is formed from a first basecoat composition. A second basecoat layer may be applied over at least a portion of the first basecoat layer, where the second basecoat layer is formed from a second basecoat composition. The second basecoat layer may be applied after the first basecoat composition has been cured to form the first basecoat layer or may be applied in a wet-on-wet process prior to curing the first basecoat composition, after which the first and second basecoat compositions are simultaneously cured to form the first and second basecoat layers.

At least one of the first and second basecoat compositions may be the coating composition of the present disclosure. The first and second basecoat compositions may be the same composition with both the first and second basecoat compositions including the coating composition of the present disclosure. The first and second basecoat compositions may be different with only one of the first and second basecoat compositions including the coating composition of the present disclosure.

The multi-layer coating system may include a primer coating layer formed from a primer composition applied over the substrate. The first basecoat layer may be positioned over at least a portion of the primer coating layer.

The multi-layer coating system may include a topcoat layer formed from a topcoat composition applied over the substrate. The topcoat composition may be applied over at least a portion of the second basecoat layer. The topcoat may be a clearcoat.

A substrate having a multi-layer coating system applied thereover may be prepared by applying a first basecoat composition onto at least a portion of the substrate and applying a second basecoat composition directly onto at least a portion of the first basecoat composition. The first and second basecoat compositions may be cured simultaneously to form first and second basecoat layers. The first and second basecoat compositions may be cured at a temperature of 100° C. or less, such as 80° C. or less, to form the first and second basecoat layers. At least one of the first and second basecoat compositions may include the coating composition of the present disclosure.

Preparing the multi-layer coating system may include forming a primer coating layer over at least a portion of the substrate and applying the first basecoat composition onto at least a portion of the primer coating layer.

Preparing the multi-layer coating system may include applying a topcoat composition onto at least a portion of the second basecoat composition. The topcoat composition may be applied onto the second basecoat composition prior to or after curing the first and second basecoat compositions. The first basecoat composition, the second basecoat composition, and the topcoat composition may be simultaneously cured at a temperature of 100° C. or less, such as 80° C. or less.

Preparing a substrate may include applying the coating composition of the present disclosure onto at least a portion of the substrate and curing the coating composition at a temperature of 100° C. or less, such as 80° C. or less, for less than or equal to 1 hour, such as less than or equal to 30 minutes, to form a cured coating layer

The coating composition may be used to prepare a coated substrate at low temperatures, such as 100° C. or less or 80° C. or less. The coating composition may be used to prepare a coated substrate at low temperatures by applying the coating composition to a substrate and curing the coating composition at low temperatures to form a coating layer over the substrate (the coated substrate).

### EXAMPLES

The following examples are presented to demonstrate the general principles of the disclosure. The disclosure should not be considered as limited to the specific examples presented.

### Comparative Example 1

Preparation of a hydroxyl, carbazate-functional polyurethane resin from hydrazine

To a four-necked round bottom flask fitted with a thermocouple, mechanical stirrer, and condenser under N₂ blanket: 136.6 grams of dipropylene glycol dimethyl ether (PROGLYDE DMM commercially available from Dow Chemical Company (Midland, MI)), 14.1 grams of dimethylol propionic acid (DMPA, commercially available from Perstorp (Malmö, Sweden)), 93.4 grams of isophorone diisocyanate (IPDI commercially available from Covestro AG (Leverkusen, Germany)) was charged into the flask and heated to 70° C. At 70° C., 0.5 grams of dibutyl tin dilaurate (DBTDL commercially available from Akzo Nobel (Amsterdam, Netherlands)) was charged into the flask. Immediate exotherm was observed. After exotherm subsided, the mixture was heated to 90° C. and held for 60 minutes until the isocyanate equivalent weight measured was 392.1 eq/g by titration (determined using a Metrohm 888 Titrando; titration by dissolving a sample (˜2.00g) of the mixture in 30 mL of a solution including 20 mL of dibutylamine and 980 mL of N-methyl pyrrolidone, followed by titration with 0.2 N HCl solution in isopropanol titration agent). At 90° C., 37.2 grams of glycerol carbonate (commercially available from Innospec Inc. (Littleton, CO)) was added and followed by a rinse with 10.5 grams of PROGLYDE DMM. The mixture was held at 90° C. for 30 minutes. After holding, 15.5 grams of trimethylolpropane (TMP commercially available from Lanxess Corp (Cologne, Germany)) was added into reaction mixture and the reaction mixture was held at 90° C. until IR spectroscopy showed the absence of the characteristic NCO band. Then, a mixture of dimethylethanolamine (DMEA, 9.4 g) and 25.3 g of 35% hydrazine in water (commercially available from Sigma Aldrich (Saint Louis, MO)) was added into reaction mixture over 30 minutes and followed by a rinse with 17.9 grams of DOWANOL PM. The reaction mixture was held at 90° C. until IR spectroscopy showed the absence of the characteristic cyclic carbonate band. Then the reaction temperature was lowered to 70° C. and 314.9 g of DI water (70° C.) was added into reaction mixture over 30 minutes. The final urethane dispersion was held at 70° C. for 30 minutes and poured out. The final dispersion had a pH of 7.97, a nonvolatile (solids) content of 26.46%, and a hydrazine content of 0.826% measured by high performance liquid chromatography (HPLC).

Non-volatile contents (also referred to herein a solids content) were measured by comparing initial sample weights to sample weights after exposure to 110° C. for 1 hour. The pH was measured herein according to ASTM D4584. Hydrazine content was determined by HPLC, which is based on the derivatization of hydrazine with 4-hydroxybenzaldehyde into the corresponding hydrazone derivative and subsequent separation using a C18 column and methanol/aqueous gradient mobile phase with UV detection at 340 nm.

### Example 2

Synthesis of a Blocked Carbazate from Propylene Carbonate and Hydrazine

To a four necked round bottom flask fitted with a thermocouple, mechanical stirrer, and condenser under N₂ blanket, 127.33 grams of propylene carbonate (commercially available from BASF (Ludwigshafen, Germany)) was charged into the flask and heated to 40° C. At 40° C., 108.50 grams of 35% hydrazine was charged into the flask over 2 hours. The mixture was heated to 50° C. and held until held at 50° C. until IR spectroscopy showed the absence of the characteristic cyclic carbonate band. Then, 356.0 grams of methyl isobutyl ketone (MIBK) was added into reaction mixture. The reaction mixture was heated to reflux and Dean stark trap was installed to remove water via azeotrope distillation. The final MIBK protected carbazate had a nonvolatile content of 26.5% and a hydrazine content of less than 10 ppm.

### Example 3

Preparation of Carbazate Functional Polyurethane Resin from Blocked Carbazate

To a four-necked round bottom flask fitted with a thermocouple, mechanical stirrer, and condenser under N₂ blanket: 52.5 grams of methyl ethyl ketone (MEK), 14.1 grams of dimethylol propionic acid (DMPA), and 93.3 grams of isophorone diisocyanate (IPDI) was charged into the flask and heated to 70° C. At 70° C., 0.5 grams of dibutyl tin dilaurate (DBTDL) was charged into the flask. Immediate exotherm was observed. After exotherm subsided, the mixture was heated to 90° C. An additional 11.9 grams of MIBK was added into reaction mixture to completely dissolve DMPA. Then, the reaction mixture was held for 30 minutes and 152.4 grams of the blocked carbazate of Example 2 was added into reaction mixture followed by a rinse with 10.5 grams of MEK. The reaction mixture was held at 90° C. until the isocyanate equivalent weight measured was 1172.8 eq/g by titration. At 90° C., 14.6 grams of trimethylolpropane (TMP) was added into reaction mixture and the reaction mixture was held at 90° C. until IR spectroscopy showed the absence of the characteristic NCO band.

Then, 9.4 grams of dimethylethanolamine (DMEA) was added into reaction mixture over 30 minutes, followed by a mixture of 0.02 g phenylphosphonic acid in 105 grams of DI water. Then, the reaction temperature was heated to reflux and held at reflux for 30 minutes. A 3-way Dean Stark distillation was set up to remove the solvent and return water back to the reactor. The final dispersion had a pH of 7.85, a nonvolatile content of 28.80%, and a hydrazine content of 0.04%.

### Example 4

Synthesis of a Blocked Carbazate from Ethylene Carbonate and Hydrazine

To a four necked round bottom flask fitted with a thermocouple, mechanical stirrer, and condenser under N₂ blanket, 216.25 grams of ethylene carbonate (commercially available Sigma Aldrich) was charged into the flask and heated to 40° C. At 40° C., 213.63 grams of 35% hydrazine solution in water (commercially available from Sigma Aldrich) was charged into the flask over 2 hours. The mixture was heated to 50° C. and held until held at 50° C. until IR spectroscopy showed the absence of the characteristic cyclic carbonate band. Then, 700.99 grams of methyl isobutyl ketone (MIBK) was added into reaction mixture. The reaction mixture was heated to reflux and Dean Stark trap was set up to remove water via azeotrope distillation. The final MIBK protected carbazate has a nonvolatile content of 42.90%, and a hydrazine content of less than 10 ppm measured by HPLC.

### Example 5

Preparation of Carbazate Functional Polyurethane Resin from Blocked Carbazate

To a four necked round bottom flask fitted with a thermocouple, mechanical stirrer, and condenser under N₂ blanket: 119.39 grams of methyl ethyl ketone (MEK), 32.03 grams of dimethylol propionic acid (DMPA), 337.71 grams of the blocked carbazate of Example 4, and 212.32 grams of isophorone diisocyanate (IPDI) was charged into the flask and heated to 80° C. At 80° C., 0.24 grams of dibutyl tin dilaurate (DBTDL) was charged into the flask. Immediate exotherm was observed. After exotherm subsided, the mixture was heated to 90° C. The reaction mixture was held at 90° C. until the isocyanate equivalent weight measured was 1080 eq/g by titration. At 90° C., 32.84 grams of trimethylolpropane (TMP) was added into reaction mixture and followed by a rinse with 23.88 grams of MEK. The reaction mixture was held at 90° C. until IR spectroscopy showed the absence of the characteristic NCO band.

Then, a mixture of 21.27 grams of dimethylethanolamine (DMEA) and 477.55 grams of DI water was added into reaction mixture and followed by 0.5 g of phenylphosphonic acid. Then, the reaction temperature was heated to reflux and held at reflux for 30 minutes. A 3-way Dean Stark distillation was set up to remove the solvent and return water back to the reactor. The final dispersion had a pH of 7.53, a nonvolatile content of 29.00% and a hydrazine content of 0.03% measured by HPLC.

The hydrazine content of the crosslinker compositions in Examples 3 and 5 was lower than the hydrazine content of the crosslinker composition of Comparative Example 1.

### Examples 6-9

### Preparation and Testing of Coating Compositions

In Examples 7-9, the cure response of a keto-functional resin with carbazate-functional crosslinker resins were measured by adhesion, paint stability, and humidity resistance methods. In Example 6, a control in which the crosslinker is adipic acid dihydrazide (ADH) is provided. For the coating compositions of Examples 6-9, the equivalent ratio of ketone to carbazate was maintained at the ratio of 1:1.

First, at ambient temperature, the keto-functional resin was mixed well with the crosslinker at a 1:1 keto: nucleophilic group ratio based on resin solids, keto equivalent weight, and nucleophilic group equivalent weight. The mixture was stirred in an 8 oz. plastic cup with an overhead mixer. Once fully blended, the coating formulation was allowed to sit under ambient conditions for at least 1 hour, but not more than 24 hours. At this time, an aliquot of the formulation was removed for paint stability measurements. A different aliquot of the formulations were drawn down onto 4×12 inch steel panels that were pre-coated with an ED 7400 electrocoat (an electrocoat commercially available from PPG Industries Inc. (Pittsburgh, PA)) using a drawdown bar. The wet films were flashed at ambient conditions for up to 10 minutes before being baked for 30 minutes at 80° C. in an oven to form a coating layer having a thickness of 15-25 microns. After baking, the panels were taken out of the oven and cooled down to ambient temperature before the Adhesion and Humidity Resistance Test. The results of the various tests is shown in Table 1. Amounts reported in Table 1 are in grams.

TABLE 1

| | Resin and Crosslinker Examples | | | | | | MEK |
|---|---|---|---|---|---|---|---|
| | Ketone | | | | | Crosslinker | Hammer |
| | Functional | Adipic acid | Comp. | | | Free Hydrazine | Rubs |
| Sample | Resin¹ | dihydrazide | Ex. 1 | Ex. 3 | Ex. 5 | level (wt. %) | (Wypall)⁵ |
| Ketone | 40 | — | — | — | — | — | 17 |
| Functional | | | | | | | |
| Resin¹ | | | | | | | |
| Comparative | 40 | 0.6 | — | — | — | — | >150 |
| Example 6 | | | | | | | |
| (Control) | | | | | | | |
| Comparative | 40 | — | 14.7 | — | — | 0.826 | >150 |
| Example 7 | | | | | | (Comp. Ex. 1) | |
| Example 8 | 40 | — | — | 16.2 | — | 0.04 | 57 |
| | | | | | | (Example 3) | |
| Example 9 | 40 | — | — | — | 10.9 | 0.03 | 56 |
| | | | | | | (Example 5) | |

| | | | After | | |
|---|---|---|---|---|---|
| | | Cross | Water Soak | After | After |
| | Viscosity | hatch | Cross hatch | Water Soak | Water Soak |
| Sample | Change² | Adhesion³ | Adhesion³ | Blushing⁴ | Blistering⁴ |
| Ketone | <25 | 1 | 0 | 4 | 1 |
| Functional | | | | | |
| Resin¹ | | | | | |
| Comparative | >150 | 3 | 0 | 1 | 3 |
| Example 6 | | | | | |
| (Control) | | | | | |
| Comparative | >150 | 5 | 5 | 0 | 0 |
| Example 7 | | | | | |
| Example 8 | 55 | 5 | 5 | 0 | 0 |
| Example 9 | 32 | 5 | 5 | 0 | 0 |

¹A latex having keto functional core-shell particles as prepared in US 2020/0290086 A1, Example 3
²Paint stability test (Viscosity Change): the viscosity of resin-crosslinker mixture was measured by CAP 2000+ viscometer 1 hour after mixing. And then the mixture was stored at 40° C. for 28 days, the viscosity was measured again. The difference of viscosity was recorded.
³Cross-hatch adhesion: ASTM D3359, test method B was performed on the coated and cured test panels. Adhesion results are assessed on a 0 to 5 scale [where 0: greater than 65% area removed and 5: 0% area removed]. In certain instances, the test panels were subjected to a 48 hour water soak at 63° C. with de-ionized water, removed from the water soak, allowed to recover for 1 hour, and then tested for cross-hatch adhesion again.
⁴Humidity resistance test: the test panels were subjected to a 48 hour water soak at 63° C. with de-ionized water, removed from the water soak, allowed to recover for 5 minutes and rated by blushing and blistering. Blushing and blistering results are assessed on a 0 to 3 scale [where 0: No visible blushing or blistering and 3: extreme haze or whitening or blistering of the coating].
⁵The Solvent Resistance Test from Table 1 (MEK Hammer Rubs (Wypall)) was performed on each cured coating composition using the following method. Methyl ethyl ketone was used as the solvent for the testing:
1. Place the test panel on a flat table or other suitable flat firm surface.
2. Fold a Wypall brand 03086 wipes commercially available at Kimberly-Clark Professional Inc. (Irving, TX) four times (yielding a section with 8 layers of wipe) and secure over the ball end of a 1000-g Ball-Peen hammer. The wipe should be snugly held in place with a rubber band in such a fashion that no wrinkles would be formed.
3. Saturate the cloth with the appropriate solvent for the material being tested, wipe should be re-saturated every 25 double rubs.
4. Immediately rub the saturated wipe over the test area using a back and forth stroke of ~4-6 inches.
5. Do not exert any downward or upward pressure on the hammer handle. The weight of the hammer controls the downward pressure.
6. Continue this back and forth action counting one “double rub” for each forward and backward motion completed until bare substrate is exposed in the center of the strip where the rubs are performed.
7. Record the test result as the number of double rubs required to expose bare substrate in the center of the rub strip.
The area of wipe should be rotated for the next test set. The wipe used for testing should always be rotated to a fresh spot every time a new area is tested, you can typically get 4 test areas on a single wipe.

The coating compositions prepared using the crosslinker prepared from a blocked carbazate showed less viscosity change and compared to the Control (ADH crosslinker) and the carbazate-functional crosslinker not prepared from a blocked carbazate. The coating compositions prepared using the crosslinker prepared from a blocked carbazate also showed excellent cross-hatch adhesion and humidity resistance properties as well as the carbazate-functional compositions of Examples 3 and 5 being essentially free of hydrazine (no more than 0.04 wt. %).

### Example 10

Synthesis of Protected Carbazate Diol from Glycerol Carbonate and Hydrazine

To a four necked round bottom flask fitted with a thermocouple, mechanical stirrer, and condenser under N₂ blanket, 283.26 grams of glycerol carbonate (Commercially available from Innospec) was charged into the flask and heated to 40° C. At 40° C., 208.67 grams of 35% hydrazine was charged into the flask over 30 minutes. The mixture was heated to 50° C. and held until held at 50° C. until IR spectroscopy showed the absence of the characteristic cyclic carbonate band. Then, 684.72 grams of methyl isobutyl ketone (MIBK) was added into reaction mixture. The reaction mixture was heated to reflux and Dean stark trap was installed to remove water via azeotrope distillation. The final MIBK protected carbazate diol had a nonvolatile content of 46.40% and a hydrazine content of less than 10 ppm.

### Example 11

Synthesis of Self-Crosslinkable Latex with Carbazate and Ketone Functionalities

Part A: A polyurethane was first prepared by charging the following components into a four necked round bottom flask fitted with a thermocouple, mechanical stirrer, and condenser: 101.7 grams of butyl acrylate, 41.2 grams of protected carbazate diol from example 10, 41 grams of FOMREZ 66-56 (hydroxyl terminated saturated linear polyester polyol, commercially available from Chemtura), 41.0 grams of POLYMEG® 2000 polyol (polytetramethylene ether glycol, commercially available from LyondellBasell), 0.6 grams of 2,6-di-tert-butyl 4-methyl phenol, 7.8 grams of hydroxy ethyl methacrylate (HEMA), and 31.5 grams of dimethylol propionic acid (DMPA). The mixture was heated to 50° C. and held for 15 minutes. Next, 118.6 grams of isophorone diisocyanate was charged into the flask over 10 minutes and mixed for 15 minutes. After mixing, 7.3 grams of butyl acrylate, 1.6 grams of triethylamine, and 0.3 grams of dibutyl tin dilaurate (DBTDL) was charged into the flask and immediate exotherm was observed. After exotherm subsided, the mixture was heated to 90° C. and held for 60 minutes. The mixture was cooled to 70° C. and 101.7 grams of butyl acrylate and 17.8 grams of hexanediol diacrylate were charged into the flask. The resulting mixture was kept at 60° C. before being dispersed into water.

Part B: A latex comprising polyurethane-acrylic core-shell particles with urea linkages, urethane linkages, pendant carboxylic acid functionality, and pendant keto and carbazate functionalities on the polyurethane shell was prepared by charging the following components into a four necked round bottom flask fitted with a thermocouple, mechanical stirrer, and condenser: 480.0 grams of deionized water, 43.0 grams of diacetone acrylamide, 20.7 grams of dimethyl ethanolamine, and 8.7 grams of ethylenediamine. The mixture was heated to 70° C. and held for two hours with an N₂ blanket. After heating the mixture, 385 grams of deionized water and 8.0 grams of AEROSOL OT-75 (surfactant, commercially available from Cytec) were charged into the flask and held at 50° C. for 15 minutes. Next, 420.0 grams of the polyurethane prepared in Part A was dispersed into the flask over 20 minutes and mixed for an additional 15 minutes. A mixture of 0.7 grams of ammonium persulfate and 60.0 grams of deionized water was then charged into the flask over 15 minutes. The temperature rose from 50° C. to 70° C. due to polymerization exotherm. The mixture was held at 75° C. for an additional hour. After being cooled to 40° C., 0.1 grams of foammaster MO 2111 NC (commercially available from BASF), 2.4 grams of ACTICIDE MBS (microbiocide formed of a mixture of 1,2-benzisothiazolin-3-one and 2-methyl-4-isothiazolin-3-one, commercially available from Thor GmbH), and 11.0 grams of deionized water were charged and mixed for an additional 15 minutes. The resulting latex had a solid content of 30.99% and an average particle size of 85.06 nm. The average particle size was determined with a Zetasizer Nano ZS following the instructions in the Zetasizer Nano ZS ZS “Making Size Measurements” and “Software” Manuals. For the particle size measurement, all glassware was washed with ultra-filtered DI to remove dust or contamination. Then samples were diluted using a dilution factor of approximately 1:1000 using ultra-filtered DI water. The Zetasizer Nano ZS was set such that the RI was 1.59 with an absorption index of 0.01; the wavelength for the light scattering detector was 632.8 nm. Ensuring no air bubbles were introduced during sample transfer to cuvette, the samples were then analyzed.

The hydrazine content was 0.001% which was measured by HPLC.

### Examples 12-13

### Preparation and Testing of Self-Crosslinking Coating Compositions

In Examples 12-13, the cure response of a self-crosslinkable resin with both ketone and carbazate functionality were measured by adhesion, paint stability, and humidity resistance methods. In Example 13, the self-crosslinkable resin was additionally mixed with a crosslinker, adipic acid dihydrazide (ADH).

Example 13: first, at ambient temperature, the self-crosslinker latex was mixed well with ADH. The mixture was stirred in an 8 oz. plastic cup with an overhead mixer. Once fully blended, the coating formulation was allowed to sit under ambient conditions for at least 1 hour, but not more than 24 hours. For both Example 12 and 13, an aliquot of the formulation was then removed for paint stability measurements. A different aliquot of the formulations were drawn down onto 4×12 inch steel panels that were pre-coated with an ED 7400 electrocoat (an electrocoat commercially available from PPG Industries Inc. (Pittsburgh, PA)) using a drawdown bar. The wet films were flashed at ambient conditions for up to 10 minutes before being baked for 30 minutes at 80° C. in an oven to form a coating layer having a thickness of 15-25 microns. After baking, the panels were taken out of the oven and cooled down to ambient temperature before the Adhesion and Humidity Resistance Test. The results of the various tests are shown in Table 2. Amounts reported in Table 2 are in grams.

TABLE 2

| | | | | Self- | | | | | |
|---|---|---|---|---|---|---|---|---|---|
| | Resin and Crosslink Composition | | | crosslinkable | MEK | | After | | |
| | Ketone | | | latex Free | Hammer | | Water Soak | After | After |
| | Functional | | Adipic Acid | Hydrazine | Rubs | Viscosity | Crosshatch | Water Soak | Water Soak |
| | Resin¹ | Ex. 11 | Dihyrdazide | level (wt %) | (Wypall)⁵ | Change² | Adhesion³ | Blushing⁴ | Blistering⁴ |
| Ketone | 40 | — | — | — | 17 | >150 | 0 | 4 | 1 |
| Functional Resin | | | | | | | | | |
| Comparative | 40 | — | 0.6 | — | >150 | >150 | 1 | 1 | 3 |
| Example 6 | | | | | | | | | |
| Example 12 | — | 30 | — | 0.001 | 23 | −2.9 | 5 | 0 | 0 |
| Example 13 | — | 40 | 0.47 | 0.001 | >150 | 31 | 5 | 0 | 0 |

¹A latex having keto functional core-shell particles as prepared in US 2020/0290086 A1, Example 3
²Paint stability test (Viscosity Change): the viscosity of resin-crosslinker mixture was measured by CAP 2000+ viscometer 1 hour after mixing. And then the mixture was stored at 40° C. for 28 days, the viscosity was measured again. The difference of viscosity was recorded.
³Cross-hatch adhesion: ASTM D3359, test method B was performed on the coated and cured test panels. Adhesion results are assessed on a 0 to 5 scale [where 0: greater than 65% area removed and 5: 0% area removed]. In certain instances, the test panels were subjected to a 48 hour water soak at 63° C. with de-ionized water, removed from the water soak, allowed to recover for 1 hour, and then tested for cross-hatch adhesion again.
⁴Humidity resistance test: the test panels were subjected to a 48 hour water soak at 63° C. with de-ionized water, removed from the water soak, allowed to recover for 5 minutes and rated by blushing and blistering. Blushing and blistering results are assessed on a 0 to 3 scale [where 0: No visible blushing or blistering and 3: extreme haze or whitening or blistering of the coating].
⁵The Solvent Resistance Test from Table 1 (MEK Hammer Rubs (Wypall)) was performed on each cured coating composition using the following method.
Methyl ethyl ketone was used as the solvent for the testing:
1. Place the test panel on a flat table or other suitable flat firm surface.
2. Fold a Wypall brand 03086 wipes commercially available at Kimberly-Clark Professional Inc. (Irving, TX) four times (yielding a section with 8 layers of wipe) and secure over the ball end of a 1000-g Ball-Peen hammer. The wipe should be snugly held in place with a rubber band in such a fashion that no wrinkles would be formed.
3. Saturate the cloth with the appropriate solvent for the material being tested, wipe should be re-saturated every 25 double rubs.
4. Immediately rub the saturated wipe over the test area using a back and forth stroke of ~4-6 inches.
5. Do not exert any downward or upward pressure on the hammer handle. The weight of the hammer controls the downward pressure.
6. Continue this back and forth action counting one “double rub” for each forward and backward motion completed until bare substrate is exposed in the center of the strip where the rubs are performed.
7. Record the test result as the number of double rubs required to expose bare substrate in the center of the rub strip.
The area of wipe should be rotated for the next test set. The wipe used for testing should always be rotated to a fresh spot every time a new area is tested, you can typically get 4 test areas on a single wipe.

The coating compositions prepared using the self-crosslinkable resin alone (Example 12) and with crosslinker (Example 13) showed less viscosity change as compared to the Controls. The coating compositions prepared using the self-crosslinkable resin alone and with crosslinker also showed excellent cross-hatch adhesion and humidity resistance properties compared to the control example.

Whereas particular embodiments of this disclosure have been described above for purposes of illustration, it will be evident to those skilled in the art that numerous variations of the details of the present disclosure may be made without departing from the disclosure as defined in the appended claims.

1. A carbazate-functional compound, comprising:
a plurality of groups comprising the following structure: 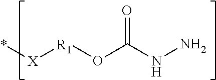
wherein X forms at least a portion of: a urethane linkage, an ester linkage, or an ether linkage,
wherein at least one R₁ from the plurality of groups is free of a hydroxyl-functional group, and
optionally, wherein at least one R₁ from the plurality of groups is a residue of carbonate, such as cyclic carbonate, and contains a same number of hydroxyl groups as the carbonate of which the at least one R₁ is a residue.

2. The carbazate-functional compound according to claim 1 as part of a composition,
wherein the composition is substantially free of residual hydrazine.

3. A The carbazate-functional compound of claim 1,
wherein X forms at least a portion of a linkage comprising a urethane linkage, an ester linkage, an ether linkage and combinations thereof.

4.-7. (canceled)

8. A coating composition comprising the carbazate-functional compound of claim 1.

9. The coating composition of claim 8, further comprising an aqueous medium into which the carbazate-functional compound is dispersed, wherein the carbazate-functional compound comprises a carboxyl group or an ammonium salt.

10. A The carbazate-functional compound according to claim 1 comprising a hydroxyl-functional blocked carbazate compound, comprising:
(a) a blocked carbazate-functional group; and
a hydroxyl-functional group,
wherein the blocked carbazate compound is a hydroxyl-functional blocked carbazate optionally comprising the following structure: 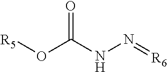
wherein R5 comprises a compound comprising a hydroxyl group,
wherein R6 comprises a carbon atom-containing group having a carbon atom double bonded to the nitrogen,
optionally, prepared by:
reacting a cyclic carbonate, with hydrazine to form an adduct; and
reacting the adduct with a ketone and/or aldehyde-functional compound to form the hydroxyl-functional blocked carbazate; or
(b) a blocked carbazate-functional group; and
a linkage, X, wherein X forms at least a portion of a urethane linkage, optionally comprising the following structure: 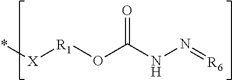
wherein R₆ comprises a carbon atom-containing group having a carbon atom double bonded to the nitrogen, optionally prepared by:
reacting a hydroxyl-functional blocked carbazate compound with an isocyanate-functional compound, or reacting a cyclic carbonate, with hydrazine to form an adduct; and
reacting the adduct with a ketone and/or aldehyde-functional compound to form the hydroxyl-functional blocked carbazate; or
(c) a blocked carbazate-functional group; and
a linkage, X, wherein X forms at least a portion of an ester linkage, optionally
comprising the following structure: 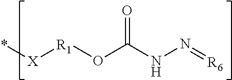
wherein R₆ comprises a carbon atom-containing group having a carbon atom double bonded to the nitrogen, optionally prepared by:
reacting a hydroxyl-functional blocked carbazate compound with an anhydride-functional compound, or
reacting a cyclic carbonate, with hydrazine to form an adduct; and
reacting the adduct with a ketone and/or aldehyde-functional compound to form the hydroxyl-functional blocked carbazate.
(d) a blocked carbazate-functional group; and
a linkage, X, wherein X forms at least a portion of an ether linkage, optionally comprising the following structure: 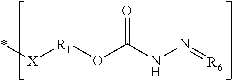
wherein R₆ comprises a carbon atom-containing group having a carbon atom double bonded to the nitrogen, optionally prepared by:
reacting a hydroxyl-functional blocked carbazate compound with an epoxy-functional compound and/or an aminoplast linkage, or
reacting a carbonate, such as a cyclic carbonate, with hydrazine to form an adduct; and
reacting the adduct with a ketone and/or aldehyde-functional compound to form the hydroxyl-functional blocked carbazate.

11.-26. (canceled)

27. A The carbazate-functional compound according to claim 1 comprising a compound, formed by:
reacting a cyclic carbonate, with hydrazine to form an adduct;
reacting the adduct with a ketone and/or aldehyde to form a hydroxyl-functional blocked carbazate; and
reacting the hydroxyl-functional blocked carbazate: (1) with an isocyanate-functional compound, (2) with an anhydride-functional compound, (3) with an epoxy-functional compound, (4) with an aminoplast linkage, to form a blocked carbazate-functional compound and/or (5) in a Michael addition reaction to form a blocked carbazate-functional compound.

28. (canceled)

29. (canceled)

30. A The coating composition according to claim 8, comprising:
a first compound;
a second compound different from the first compound, wherein the second compound comprises the carbazate-functional compound; and
optionally wherein the first compound is reactive with a carbazate-functional group of the carbazate-functional compound;
optionally a self-crosslinkable molecule or particle comprising at least one carbazate-functional group and at least one second functional group, reactive with a carbazate-functional group and
optionally wherein the coating composition comprises an aqueous medium.

31. (canceled)

32. The coating composition of claim 30, wherein the first compound comprises a ketone-functional, aldehyde-functional, isocyanate-functional, epoxy-functional, and/or double bond-functional group; and
optionally wherein the first compound and/or the second compound comprises a polymer and/or an oligomer; and
optionally wherein the first compound is bonded to the second compound by reacting the carbazate-functional group of the second compound with a functional group of the first compound.

33.-34. (canceled)

35. The coating composition of claim 30, wherein:
the first compound comprises a precursor of an acrylic core formed from acrylic monomers; and
the second compound comprises a precursor of a carbazate-functional shell comprising the carbazate-functional compound, wherein the shell at least partially encapsulates the core to form a core-shell structure; and
optionally wherein the core-shell structure is formed by reacting the precursor of the carbazate-functional shell with the acrylic monomer from the precursor of the acrylic core, wherein the core-shell structure comprises carbazate functionality on the core and/or the shell and/or the core-shell structure comprises ketone and/or aldehyde functionality on the core and/or the shell.

36. (canceled)

37. The coating composition of claim 35, wherein the first compound and/or the second compound comprises a carboxyl group.

38. (canceled)

39. A The coating composition according to claim 8, comprising:
a self-crosslinkable molecule or particle comprising at least one carbazate-functional group and at least one second functional group, reactive with a carbazate-functional group and optionally a crosslinker; and
optionally wherein the second functional group and/or optional compound comprise a ketone-functional, aldehyde-functional, isocyanate-functional, epoxy-functional, and/or double bond-functional group; and
optionally wherein the self-crosslinkable molecule or particle comprises a polymer and/or an oligomer.

40.-41. (canceled)

42. A substrate at least partially coated with a coating formed from the coating composition of claim 8, optionally wherein the substrate comprises a vehicle substrate.

43. (canceled)

44. The substrate according to claim 42, coated by a process, comprising:
applying the coating composition of any of claim 8 onto at least a portion of the substrate; and
curing the coating composition at a temperature of 100° C. or less for less than or equal to 1 hour to form a cured coating layer.

45. The carbazate-functional compound of claim 1 in a solution, comprising:
the blocked carbazate compound; and
a ketone and/or aldehyde-containing solvent,
wherein a blocking group of the blocked carbazate compound is derived from the ketone-containing solvent; and
optionally wherein the ketone-containing solvent comprises: methyl isobutyl ketone (MIBK), methyl ethyl ketone (MEK), acetone, and/or mixtures or combinations thereof.

46. (canceled)

47. The carbazate-functional compound according to claim 1, comprising a (thio)carbazate-functional compound, comprising:
a plurality of groups comprising the following structure: 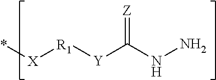
wherein X forms at least a portion of: a (thio)urethane linkage, a (thio)ester linkage, or a (thio)ether linkage,
wherein each Y and Z is independently selected from O and S,
wherein at least one R₁ from the plurality of groups is free of a hydroxyl-functional group.

48. The coating composition according to claim 8, comprising:
a first compound; and
a second compound different from the first compound, wherein the second compound comprises a (thio)carbazate-functional compound comprising:
a plurality of groups comprising the following structure: 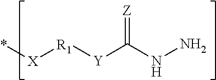
wherein X forms at least a portion of: a (thio)urethane linkage, a (thio)ester linkage, or a (thio)ether linkage,
wherein each Y and Z is independently selected from O and S,
wherein at least one R₁ from the plurality of groups is free of a hydroxyl-functional group.

49. (canceled)

50. The substrate according to claim 42 at least partially coated with a coating formed from a coating composition comprising:
a first compound; and
a second compound different from the first compound, wherein the second compound comprises the a (thio)carbazate-functional compound comprising:
a plurality of groups comprising the following structure: 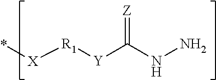
wherein X forms at least a portion of: a (thio)urethane linkage, a (thio)ester linkage, or a (thio)ether linkage,
wherein each Y and Z is independently selected from O and S,
wherein at least one R₁ from the plurality of groups is free of a hydroxyl-functional group, wherein the substrate optionally comprises a vehicle substrate.

51. (canceled)

52. The substrate according to claim 44 coated using a A process further comprising:
applying the coating composition comprising:
a first compound; and
a second compound different from the first compound, wherein the second compound comprises the a (thio)carbazate-functional compound comprising:
a plurality of groups comprising the following structure: 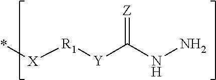
wherein X forms at least a portion of: a (thio)urethane linkage, a (thio)ester linkage, or a (thio)ether linkage,
wherein each Y and Z is independently selected from O and S,
wherein at least one R₁ from the plurality of groups is free of a hydroxyl-functional group, wherein the substrate optionally comprises a vehicle substrate
onto at least a portion of the substrate; and
curing the coating composition at a temperature of 100° C. or less for less than or equal to 1 hour to form a cured coating layer.

53. The carbazate-functional compound of claim 45 comprising:
a blocked (thio)carbazate compound; and
a ketone-containing solvent,
wherein a blocking group of the blocked (thio)carbazate compound is derived from the ketone-containing solvent optionally, wherein the ketone-containing solvent comprises: methyl isobutyl ketone (MIBK), methyl ethyl ketone (MEK), acetone, and/or mixtures or combinations thereof.

54. (canceled)
